SEQUENCE LISTING (52 sequences)

SEQ ID NO: 1 - DNA, 2814 bp, C. elegans clk-2 cDNA
ctcaagatgaatttacgaagtcgcctggtaaatgccacggaacgtgctgtgctttttcaaattttcaaagatgtgcagaatgatccggaaaagtacgacaatgcagttgaggcgatctgtgaatcaatcgactattttggcaaatttttgaccgatagcgagtatcttacacaaatcaagccgattcttgatacacagtgcccaacaaagtcgataatttgcttctcgaaatgtttgacaaaagtgagcacagatataaatactaccacatttcgagatgtgatcaccatgctcgactggttgaagtatgtcgttgaaaaatcgctgacaagtgctatttgtagcagtctgaaagttaaagaaactgatgtcagtgcagttcagttgtatcgagaattcgcatcagcatgttcaaatattccggagaaagtttcgaattgttgtgcaaaggcattgtctggcgagcatgtcaaatatatcaacacggttaagtggatattcaaaatgaatctggtgcaaggaattcaaaaggctatgcttcttgctcacgacgacattgtaactgctgccccgttcacttcattctacggatccggtggtccttatatgaagactgtcgcagaaattatttcatctggaagaaacatagatatcaccaacaaggatgggcttctagttcaaatgattgaatggattggttcactaaacaattttgattctcaatggcgtcggatgatgtttctcatcttccaagagcccacatatcagggaattcaagttcatgaatcactactgacaacattgttcctaatttcgaaaagtgaccaaatcttgaaacgatgtatcgaagccactgatctgactggaacactgaagcgtgtagtgatggttaagctcccgtttcagcgagttctcaaacgaaagaccatcgagattcttatcaattttgtttatcgaactaaggaacaatttgccatccagctattagagacttctgtgaaaatctggagtgatctcaattacgcaaaaagtgctccagaatcacaagaacgacacatagtcagaatgatattatacttggttcatcttttcagaacatgttcttcaatcgattgggagtcactcttcctgaactctatggatggagttcattgtcgaatgagcatgttgcctatgtacgtccaaagtggtatttttgttaatcaagcactgtgcaagcaagcgacaaagcatcgatcgaaaacgcacggatcagatgagcaacctccagagactctagaagaaaacaaattcgtttcaagtgaagtgggaaaaatatggtttgaagagatgacgtcaattttggaacatggatttaattcttctacagtgaaagattctgagcgagttcgagaaaccgccaacgaaataaccaaagacgattcgggtgaagaatttgaagaaaccaatgcacagcgtcttcaaaacaacaaagattcggcagcaatcacatcgaaaaacaatctacgtttagattctgatgatgacgaagactttcctgactatcaagttaatgaatcagaaaagatcttcaagaatttagaaattggagaagaaccgaaaaataaagtgacacctccagcatacattgcagatgctttcgaaatgctattagagaaagaaaaatacgaggtttttgaagcagctttctttaatattacgaatttgatcaatcgccggccaattggatttccacaaatcgctgagaagttgttcatccgaatcctccatcttcaaaacaattttggaacgcctaaattcaaggaaactgttgatgaaattgcagttgcatgtatcactcagcgtccagaaattgtcccatctgtagtgcgtctgatcattgcaccaggtcaaggtttcagtatcaaacaacgtcttcttcattacattcacaatgctgctgatggaatgggtgcattggataagaaacttgaagagtgtgtaatggcgcaacaattgagaattggtggtccaacgttaagtattattcttcatcgaactataaacactgattatgacgatgaggatgaagatccccacagacttttagttcctgaatggcgacgaatggtggatgctcgcattgctgcaaataccagaagaattggaacgacgcgagagccaccaagagccggagttgtcaatcgtctcgcacaagctgccaaatatatgttttatcctttgttggttttgccacgtggtgagaacgcaagtcttttgggcaaggactccgatctactcgcctcactcatcatggttgcatcgatggtttatgtgagatgtggcgtatgtcctcaaattcatcgaatgtcaagtgagcttatatcatatgcaacgcctcatcgattctctgaaaacgcaaaactacggactgcctgcatcattgcccatctgaatgtgacgactttgttgcctggagatcttatggatgaactgtttgatgtaccggcacttattggatggtttgattgggccaattctgtactggttaatgcatcttcatcacaattggaaaaggatatgactcgccagtttggtcatagtgttacaaaacaccttcaacgttatcatccagctgtactgcaacaccaagacgtttaaatagttactattcacttgttttccttcttttcaagtactgtatcatttcttactatcttgccaacactttgatctctacctcgttcacttcttgctttgccacccgttgatatcacctgtctcattcatttatcagcatgttcataatatcaaaaataaaatcttatcaaatgt

SEQ ID NO: 2 - protein, 876 aa, C. elegans
  clk-2 protein
MNLRSRLVNATERAVLFQIFKDVQNDPEKYDNAVEAICESIDYFGKFLTDSEYLTQIKPILDTQCPTKSIICFSKCLTKVSTDINTTTFRDVITMLDWLKYVVEKSLTSAICSSLKVKETDVSAVQLYREFASACSNIPEKVSNCCAKALSGEHVKYINTVKWIFKMNLVQGIQKAMLLAHDDIVTAAPFTSFYGSGGPYMKTVAEIISSGRIDITNKDGLLVQMIEWIGSLNNFDSQWRRMMFLIFQEPTYQGIQVHESLLTTLFLISKSDQILKRCIEATDLTGTLKRVVMVKLPFQRVLKRKTIEILINFVYRTKEQFAIQLLETSVKIWSDLNYAKSAPESQERHIVRMILYLVHLFRTCSSIDWESLFLNSMDGVHCRMSMLPMYVQSGIFVNQALCKQATKHRSKTHGSDEQPPETLEENKFVSSEVGKIWFEEMTSILEHGFNSSTVKDSERVRETANEITKDDSGEEFEETNAQRLQNNKDSAAITSKNNLRLDSDDDEDFPDYQVNESEKIFKNLEIGEEPKNKVTPPAYIADAFEMLLEKEKYEVFEAAFFNITNLINRRPIGFPQIAEKLFIRILHLQNNFGTPKFKETVDEIAVACITQRPEIVPSVVRLIIAPGQGFSIKQRLLHYIHNAADGMGALDKKLEECVMAQQLRIGGPTLSIILHRTINTDYDDEDEDPHRLLVPEWRRMVDARIAANTRRIGTTREPPRAGVVNRLAQAAKYMFYPLLVLPRGENASLLGKDSDLLASLIMVASMVYVRCGVCPQIHRMSSELISYATPHRFSENAKLRTACIIAHLNVTTLLPGDLMDELFDVPALIGWFDWANSVLVNASSSQLEKDMTRQFGHSVTKHLQRHHPAVLQHQDV

SEQ ID NO: 3 - protein, 836 aa, Homo sapiens
  clk-2 protein
MEPAPSEVRLAVREAIHALSSSEDGGHIFCTLESLKRYLGEMEPPALPREKEEFASAHFSPVLRCLASRLSPAWLELLPHGRLEELWASFFLEGPADQAFLVLMETIEGAAGPSFRLMKMARLLARFLREGRLAVLMEAQCRQQTQPGFILLRETLLGKVVALPDHLGNRLQQENLAEFFPQNYFRLLGEEVVRVLQAVVDSLQGGLDSSVSFVSQVLGKACVHGRQQEILGVLVPRLAALTQGSYLHQRVCWRLVEQVPDRAMEAVLTGLVEAALGPEVLSRLLGNLVVKNKKAQFVMTQKLLFLQSRLTTPMLQSLLGHLAMDSQRRPLLLQVLKELLETWGSSSAIRHTPLPQQRHVSKAVLICLAQLGEPELRDSRDELLASMMAGVKCRLDSSLPPVRRLGMIVAEVVSARIHPEGPPLKFQYEEDELSLELLALASPQPAGDGASEAGTSLVPATAEPPAETPAEIVDGGVPQAQLAGSDSDLDSDDEFVPYDMSGDRELKSSKAPAYVRDCVEALTTSEDIERWEAALRALEGLVYRSPTATREVSVELAKVLLHLEEKTCVVGFAGLRQRALVAVTVTDPAPVADYLTSQFYALNYSLRQRMDILDVLTLAAQELSRPGCLGRTPQPGSPSPNTPCLPEAAVSQPGSAVASDWRVVVEERIRSKTQRLSKGGPRQGPAGSPSRFNSVAGHFFFPLLQRFDRPLVTFDLLGEDQLVLGRLAHTLGALMCLAVNTTVAVAMGKALLEFVWALRFHIDAYVRQGLLSAVSSVLLSLPAALLEDLMDELLEARSWLADVAEKDPDEDCRTLALRALLLLQRLKNRLLPPASP

SEQ ID NO: 4 - DNA, 3320 bp, Homo sapiens clk-2
gcgccccagaggctcaagaaaacccgcgggagcctcgcccggacccaggaactcgtgctcggggccaaccggctgggccgcgatcgcgtttcgtccggggccgcggcggccgtggggaatcggctgcagcgaatcggtggcgcgcggcgcctgagcgcgctgcagtcacccgggagccgggtccaggtcgtcttcccgtgacgcccagatctgtcctgcaggatggagccagcaccctcagaggttcgactcgccgtccgggaagccattcatgccctctcgtcttcggaggatggcggccacatcttctgcaccctggagtccctgaagcggtatctcggtgagatggagcctccagcgctcccgagggagaaggaggagtttgcctcggcccacttctcgcctgtcctcagatgtcttgccagcaggctgagcccagcctggctggagctgctgccccatggccgcctggaggagctgtgggccagcttcttcctggagggcccggcggaccaagccttcctggtgttgatggagaccatcgagggtgctgcgggccccagcttccggctgatgaagatggcgcggctgctggccagattcctgcgcgagggccggctggcagtgctgatggaggcgcagtgtcggcagcagacgcggcccggcttcatcctgctccgggagacgctgctgggcaaggtggtggccctgcccgatcacctgggcaaccgcctgcagcaggagaacttggccgagttcttcccccagaactacttccgcctgctcggcgaggaggtcgtccgggtgctgcaggcggttgtggactctctccaaggtggcctggattcctccgtgtccttcgtgtctcaggtccttgggaaagcctgtgtccacgggaggcagcaggagatcctgggcgtgctggtaccccggctggcagcgctcacccagggcagctacctgcaccagcgcgtctgctggcgcctggtggagcaagtgccggaccgggccatggaggctgtgctgaccgggctggtggaggccgcactggggcctgaggtcctttcgagactgctggggaacctggtggtgaagaacaagaaggcccagtttgtgatgacccagaagcttctgttcttacagtcccggctcacgacgcccatgctgcagagcctgctgggccatctggccatggacagccagcggcgcccgctcctgctgcaggtgctgaaggagctgttggagacgtggggcagcagcagtgccatccgccacactcccctgccgcagcagcgccacgtcagcaaggctgtcctcatctgcctggcgcaactcggggagccggaactgcgggacagccgggatgaactgctggccagcatgatggcgggcgtgaagtgccgcctggacagtagcctgccccccgtgcgacgcctgggcatgatcgtggcagaggtcgttagtgcccggatccaccccgaggggcctcccctgaaattccagtacgaagaggatgaactgagcctcgagctgctggccttggcctccccccagcctgcgggtgacggcgcctcggaggcgggcacgtccctcgttccagccacggcagagccccctgcagagacccccgcagagatcgtggatggcggcgtcccccaagcacagctggcgggctctgactcggacctggacagcgatgatgagtttgtcccctacgacatgtcgggggacagagagctgaagagcagcaaggctcctgcctacgtccgggactgcgtggaagccctgaccacgtctgaggacatagagcgctgggaggcagccctgcgggcccttgagggcctggtctacaggagccccacagccactcgggaggtgagcgtggagctggccaaggtgcttctgcatctggaggagaagacctgtgtggtgggatttgcagggctgcgccagagagccctggtggccgtcacggtcacagacccggccccggtggccgactatctgacctcacagttctatgccctcaactacagcctccggcagcgcatggacatcctggatgtgctgactctggctgcccaggagctgtctaggcctgggtgcctcgggaggactccccaacctggctccccaagtcccaacaccccgtgcctgccagaggcagccgtctctcagcctggcagtgccgtggcgtctgactggcgggtggtggtggaggagcggatcagaagcaagacccagcggctctccaagggtggcccgaggcagggcccggcaggcagccccagcagattcaactccgtggccggccacttcttcttccccctccttcagcgctttgacaggcctctggtgaccttcgacctcttgggagaagaccagctggttctcggaaggctggcgcacaccttaggggccctgatgtgcctggctgttaacaccacggtggctgtggccatgggcaaggccctgctggaattcgtgtgggcccttcgcttccacatcgatgcctacgtgcgccaggggctgttgtcggccgtctcctccgtcctgctcagcctgcctgctgcgcgcctgctggaggacctgatggacgagctgctggaagcccggtcctggctggcggacgtggctgagaaagacccggacgaggactgcaggacgctggcactgagggccctgctgcttctgcagagactcaagaacaggctcctcccacccgcgtctccctagtccctggaggcctccccaggaccaccctcgccgacagcaaggcaggcggctgagcagcggcctggagcagcagagccaggctttgtagcgaggccaggtcttcggccgcatccggtacggagagtgcagatgcaggaaggcccggcctgccgctatttatagtgcagccagtccgctaaaaatacactgggcctgggcactgcccgccgggacatggcagcctggacgtggggctggggctgtgggcgctgctggcggggttgactcttccagtgagggcagaaccaggctggcaggaggggaggacggtgtacctgctgctcagagcccccaaggctctcctctgagagccaccaagcaggacagagcagctcttgtcccaggtccctcgggctgagcgccgtgtcaccaggagaatagtgctcacagcccaggcagggtgtgtggctcctggatgggctcgtggggcgggatgggacagggcacgggctctcagaaaataaactgctttattggaaaaaaaaaaaaaaaaaaaaaaaaaaaaaaaaaaaaaaaaaaaaaaaaaaaaaaaaaaaaaaaaaaaa

SEQ ID NO: 5 - DNA, 466 bp, Mus musculus clk-2 cDNA
gtgacccgcaagctcctgctcctgcagtaccagcacacgacacccatggtgcagagcctgttggggtacttggctctagacagtcagcggcggccactcctcatacaggtgcttaaggagctgctggagacctggggctgcagcagtgctgtccgacacacacccctggagcagcagtgttacatcagcaaggccatccttgtctgcctggcacacctcggggagccggagctgcaggacatccgggatgaattgctggccagcatgatggcaggcgtgaagtgccgcctggatagcagcctgccccctgtgcgccgcttgggcatgattgtggccgaggtcatcagctccaggatccaccctgaggggcctctcctgaaattccaatatgaagatgacgagatgagccgtgagttgctggccttggctaccccagagcctgcgggtgactgctcctcggtgtcac

SEQ ID NO: 6 - DNA, 267 bp, Artificial Sequence
  part of Mus musculus cDNA
tgtcccatgtgctgacttctgaggtggctgtgctagtgggtaaggccctgctggagtttgtatgggcccttcgcttccacgttgacatttacgtgcgccggggcttgctgtctgctgtgtcctctgtcctcctcagtgtacccacagagcggctgctgggggacctgccagatgagctactggaagccagatcctggttggcagatgtggctgagaaggatgtggacgaggactgtagggagctggcagtaagggctctgctgcttc

SEQ ID NO: 7 - DNA, 337 bp, Artificial Sequence
  part of Mus musculus cDNA
tcacagttctatggcctaaactatagcctccgccagcgcatggacatcctggacgtccttgttctggctgcccaggcactgtctcggccaaagagcctgcagagacgttcccagcacggtccccccgttcctggcaccatgtgttcaccagcactagccgtttctcagactggcaatgtcgctgctcctgactggcaggtggttgtggaggagcggatcagaagcaagacccggaggttctcgaagggctgtcctcagcgggacgtgtcaggcgtccccaatgaattcagctctgtggctggctacttcttcttccccctccttcagcactttgaca

SEQ ID NO: 8 - protein, 153 aa, unknown
  part of Mus musculus clk-2 protein
VTRKLLLLQYQHTTPMVQSLLGYLALDSQRRPLLIQVLKELLETWGCSSAVRHTPLEQQCYISKAILVCLAHLGEPELQDIRDELLASMMAGVKCRLDSSLPPVRRLGMIVAEVISSRIHPEGPLLKFQYEDDEMSRELLALATPEPAGDCSS

SEQ ID NO: 9 - protein, 112 aa, unknown
  part of Mus musculus clk-2 protein
SQFYGLNYSLRQRMDILDVLVLAAQALSRPKSLQRRSQHGPPVPGTMCSPALAVSQTGNVAAPDWQVVVEERIRSKTRRFSKGCPQRDVSGVPNEFSSVAGYFFFPLLQHFD

SEQ ID NO: 10 - protein, 85 aa, unknown
  part of Mus musculus clk-2 protein
LTSEVAVLVGKALLEFVWALRFHVDIYVRRGLLSAVSSVLLSVPTERLLGDLPDELLEARSWLADVAEKDVDEDCRELAVRALLL

SEQ ID NO: 11 - protein, 350 aa, unknown
  composite protein sequence of Mus musculus clk-2
VTRKLLLLQYQHTTPMVQSLLGYLALDSQRRPLLIQVLKELLETWGCSSAVRHTPLEQQCYISKAILVCLAHLGEPELQDIRDELLASMMAGVKCRLDSSLPPVRRLGMIVAEVISSRIHPEGPLLKFQYEDDEMSRELLALATPEPAGDCSSSQFYGLNYSLRQRMDILDVLVLAAQALSRPKSLQRRSQHGPPVPGTMCSPALAVSQTGNVAAPDWQVVVEERIRSKTRRFSKGCPQRDVSGVPNEFSSVAGYFFFPLLQHFDLTSEVAVLVGKALLEFVWALRFHVDIYVRRGLLSAVSSVLLSVPTERLLGDLPDELLEARSWLADVAEKDVDEDCRELAVRALLL

SEQ ID NO: 12 - protein, 122 aa, unknown
  part of Sus Scrofa clk-2 protein
KAPVYVRDCVEALTASEDWERWEAALRALEGLVFRSPAAAREVSVELAKVLLHLEEKTAVAGFEGLRQRALVAVTVTDPARVAEYLTAQFYALNYSLRQRMDILDVLTLAAQELSRPGRLGR

SEQ ID NO: 13 - protein, 554 aa, D. melanogaster clk-2
LISLPAQVANRLGRRLPETFAPVSYQKLLLRQWLKSLHFVLQCDDNREYFDLEPYSWLLSQAINLIYDVSTLESLLRVLKDYAVAPRGRKVVHTILKELDPAACLKTAQSALSAGLNLYVLIGAATLETPHWKHCLLQKLPLQRTPVDNKQLITLASYLNAVAPAQLQVLLNQLLGIWSKRISLQKLGSQEHLAISKLLVLAQLDSDDDEPLDEDDDELKPYDMSNDTTTTIDQRPKFVIDLLHLLREKVENYQVFEGALGTAEQLIRGQLAKHDTQLALDLLQLFLVMEMQFYYEQFERTQFKCCVAICVAHPGPCAEYLCRQFHTDNSFYSASVRILILQVLAATAKELSGDENMQNEMEIVDVIPPAAKHPRKFEFQQEEESPAARLAAAQRIIRDRLRAKTKRYFSKPKAGDQMEKANPFHPVAGTFFFSLVRGQRTRQMLYVKYEIDTQLLVNLLNTMSVLVMCSQNCPLLPAMTREIFDLCAFVRFNAEARVRAATLQLIGIALVTTPAHVLAQHFAESLNELQRWLNDFIRSPLVGGETSEECRELA

SEQ ID NO: 14 - protein, 1017 aa, unknown
  A. thaliana clk-2 putative protein
MAEGTKQERTLENNLLHKVGEAVSAISDAKHVDQVISAIHSVAVLLFPVDPSLFSGNFEMLHIVRGSGTFGLLMILYLGSAQVCSSVVPSADERNEWLETFYRGVAFPTFARVLLLDVASDWLSCFPISVQKHLYDKFFLDGSVIEVVQVLVPFLHHVGDGGVNANSVQTNVERLLILCLLENDGVLKITKEIGNIYQGHNSSNGSLKPLLSRLSQILTSIPDKARASCTEANCTVIVLSFVGEVFSRICRRGLSDLLLSEVTPHVLAQVRRLLNSKIGAIEVDTFQLDPTTRIWSKTMEAVTDPYAVEKMAEQLLHQLYAEHPSDVEAFWTIWTLFHRNVIHQASVRQAKCFLWQLDSFFRYPFFFFHFHPNAVKQCVLECPPVTNTLAKGDVTQGLLETTQRLASVWSKREFLQSVQLEQQAYLQFLFPVTDISDITAALGLCLENMSREELDRTKDVMHSILQGVSCRLENPGDLVRKMASSIAFMFSKVIDPKNPLYLDDSITDNAIDWEFGLQTASITNTMENGDGENKRSASLTEVNESSRRNKQKENRKSKNISAFVLADPNEIVDLATLNCDTESDKDDGDDDASVSSDNSSVTSLEPYDLMDDDKDLGKQFTHLVDVVGALRKTDDADGVEKAIYVAEKLVRASPDELTHIAGDLARTLVQVRCSDIAIEGEEDSAEEKRQRALIALLVTRPFESLETLNNILYSPNVDVSQRIMILDVMAEAARELANSKTLKPKHEARGPLISNISDPQPWYLPSNASTPWKKVSETGSFHLNWANRFERELQSKPGQTKKGKSRRWSLKSADRDQNSTDWSQNRFPLYAAAFMLPAMKEFDKKRHGVDLLGRDFVVLGKLVHMLGVCMQCASMHPEASALAISLLDMLQRREVCNHPEAYVRRAVLFAASSVLVSLHPSYIVSTLVEGNLDLSRALEWIRTWALQIADSDIDRDCYTMALSCLQLHAEMALQTSRALESTGGSSSSSSIRPMNISLPSGISKLTSIKLPSSNVHL

SEQ ID NO: 15 - DNA, 469 bp, Artificial Sequence
  part of Oryza sativa clk-2 cDNA
gtttctactgggtttggaatggatacgcacatgggctcttccatgttgcagaaacagatcctgatacagaatgcacatcaatggctatgaccctccctgcggctttcattctgagatggcccttcagacatcacgagcactggaatcggcggatcacagcaaggccagcagcagcagcagcaggtcgctaccttctaagcttgataacatcatcataccatttgccaacatgatgtgatcgatgtactagattaagttgtaaataagcatatattatcttgccatgtaatattagaaattccaggtcagctaattctgattaggcttgatattgtcatattacagggttttttaatctgggatttgaaaatgctagaatttttgtcgattcgtgtggagattgtatggaagttgtcctgatccagattaaagagaattgatgaaaattgtacccaaaaaaaaaaaaaaa

SEQ ID NO: 16 - DNA, 376 bp, Artificial Sequence
  part of Oryza sativa clk-2 cDNA
agttcctagtgatcagggacctgctggtgcaggtctntggagggaagtctcagaatcaggaacacttctgaattggtcacaccggtatgaaagagaagttccatctagatctggtcaagttaaatcaggaaaatctcgtaaatggggtcttggaaaagctaaagatttgcagacagagtggtcaaaaaacagatttcctttatatgctgctgcttttatgctccctgttatgcaaggatatgataaaagatcacatggtgttgacttgctcaatcgggactttgttgtcctaggtaaattgatatacatgcttggtgtctgtatgaagtgcatggcaatgcatccagaagcatcagctgttgccccagctcttctt

SEQ ID NO: 17 - protein, 122 aa, unknown
  part of Oryza sativa clk-2 protein
PWREVSESGTLLNWSHRYEREVPSRSGQVKSGKSRKWGLGKAKDLQTEWSKNRFPLYAAAFMLPVMQGYDKRSHGVDLLNRDFVVLGKLIYMLGVCMKCMAMHPEASAVAPALLDMIRSRAV

SEQ ID NO: 18 - protein, 70 aa, unknown
  part of Oryza sativa clk-2 protein
LEWIRTWALPCCRNYRMHINGYDPPCGFHSEMALQTSRALESADHSKASSSSSRSLPSKLDNIIIPFANM

SEQ ID NO: 19 - protein, 192 aa, unknown
  composite protein sequence of Oryza sativa clk-2 protein
PWREVSESGTLLNWSHRYEREVPSRSGQVKSGKSRKWGLGKAKDLQTEWSKNRFPLYAAAFMLPVMQGYDKRSHGVDLLNRDFVVLGKLIYMLGVCMKCMAMHPEASAVAPALLDMIRSRAVLEWIRTWALPCCRNYRMHINGYDPPCGFHSEMALQTSRALESADHSKASSSSSRSLPSKLDNIIIPFANM

SEQ ID NO: 20 - DNA, 464 bp, Artificial Sequence
  part of Glycine max clk-2 cDNA
ttttaggaattggtcaaatagctatgagagggaacttcccccaaaacctaatcaggtcaagaaagggaaaacacgccggtggagcctacaatctcccacacaacaaaaccagatggagtattctcataataagttacccatgtatgctgctgcattcatgcttcctgccatggagggatatgataaaaaaaggcaaggtgttgacttgcttggaagagattttattgtcttggggaaactcatttatatgcttggggtctgtatgaaatctgtagccatgcatccagaagcttctatgctggctccttccctcctaaatatgttaagatccagggaggtatgccatcaccaggaagcatatgtgagaagagccgtgctttttgcagctgcatgtgtattggttgcccttcatcctacttacatttcatccaccttactcgaaggaaatgctgaaatttcgactg

SEQ ID NO: 21 - DNA, 521 bp, Artificial Sequence
  part of Glycine max clk-2 cDNA
tccggagaaggactctgattccccttccaataaagagaaaagtatttgtttaaagggtaaaaagaagttattggactttaatgcgcttgatccagatgagattattgatccagcatcactgaatcttgaatcagacgatagcgatgaggatgctgacgatggtgctagtgagaattcatattcttcaagtgattcatctttacggccatatgatttgtcagatgatgactcagatttgaaaagaaaaatttcacagttggctgatgtagttgcagctcttagaaaatccaatgatgccgatggggtggaaagggctattgatgtagctgaaaagctcataagagcatcccccgatgaactaaaacatgcagcaagggatatgaccagaactcttgttcaggttcggtgctctgatatagctttagaaggtgcagaagaatcaactgaagacaaaagacaaagatcattagttgccttagtagttacctgcccatttgaatcacttgagtcactaaacaacc

SEQ ID NO: 22 - DNA, 741 bp, Artificial Sequence
  part of Glycine max clk-2 cDNA
atnnatnnnnnntagcagaaataattagcagtttagcacgtttagaatttatattctgggctagaagctattcacaatagctagaagcatcactaatccccattttagtagggaattttaatggtcactttggatgcatcagaaggaagaacaggacctgccttgagtgaacttcttactgactccaatgctcgggaagtttgaagagccatctcggcatggagttgtatacatgtcatagccatcgtatagcattctttatctgtgtctgactcggctatgtcaagtgcccatgtgcgaatccattcaaggccagtcgaaatttcagcatttccttcgagtaaggtggatgaaatgtaagtaggatgaagggcaaccaatacacatgcagctgcaaaaagcacggctcttctcacatatgcttcctggtgatggcatacctccctggatcttaacatatttaggagggaaggagccagcatagaagcttctggatgcatggctacagatttcatacagaccccaagcatataaatgagtttccccaagacaataaaatctcttccaagcaagtcaacaccttgcctttttttatcatatccctccatggcaggaagcatgaatgcagcagcatacatgggtaacttattatgagaatactccatctggttttgttgtgtgggnnnnngtnnncnccacnnncgtgttttccctttcttgacctgattngnttttgnnnga

SEQ ID NO: 23 - DNA, 456 bp, Artificial Sequence
  part of Glycine max clk-2 cDNA
ttgaaatttcaactggccttgaatggattcgcacatgggcacttgatgtagccgagtcggacacagataaagaatgctatacgatggctatgacatgtatacagctccatgttgagatggctcttcaaacttcccgagcattggagtcagtaagaaattcactcaaggcaggtcctgttcttccttctgatgcatccaaagtgaccattaaaattccccacttaaatggggattagtgatgcttctagctattgtggatggattctagcccagaatacaaattctatatcatgctaaactgctaattatttctgctactctattaatttaatgttttatggggtccttgttttcgtaagaaattgtatattttaggattgtatatattctcacaaggggacgaggttgatgtcaatgctcccctacacacccacccttgatgtagcacgtgttact

SEQ ID NO: 24 - DNA, 455 bp, Artificial Sequence
  part of Glycine max clk-2 cDNA
aaactcgcggcggaaggcagcagagtggcgccgatggaggagggtttagagaagagagaattggaaggcgaagtagttaccaaggttgtcgaagtggtttcggctataaagaatgcgaagcacgtcgatcaagtcattcgcgcgcttcattccttagtcacccttcttttcccctttgactcttcactcctatcagatagcattgaccagagttaccgagaccaggtcgaagttccttctgcagaaaaacgacatgcttggtggcgtgcgttttatcgaggagctgcttttcctacactggctaggtttttattacttgatgttgcctcgaactggttgggttgttttccctttatggcgcagaagtacatttatgatgttttctttgttcgtggattggtcactgacgttctgcagattctggttccttttcttcagctgagtgcgagtgatgg

SEQ ID NO: 25 - protein, 208 aa, unknown
  part of Glycine max clk-2 protein
KXNQVKKGKTRXWXXXXPTQQNQMEYSHNKLPMYAAAFMLPAMEGYDKKRQGVDLLGRDFIVLGKLIYMLGVCMKSVAMHPEASMLAPSLLNMLRSREVCHHQEAYVRRAVLFAAACVLVALHPTYISSTLLEGNAEISTGLEWIRTWALDIAESDTDKECYTMAMTCIQLHAEMALQTSRALESVRSSLKAGPVLPSDASKVTIKIP

SEQ ID NO: 26 - protein, 151 aa, unknown
  part of Glycine max clk-2 protein
NWSNSYERELPPKPNQVKKGKTRRWSLQSPTQQNQMEYSHNKLPMYAAAFMLPAMEGYDKKRQGVDLLGRDFIVLGKLIYMLGVCMKSVAMHPEASMLAPSLLNMLRSREVCHHQEAYVRRAVLFAAACVLVALHPTYISSTLLEGNAEIS

SEQ ID NO: 27 - protein, 172 aa, unknown
  part of Glycine max clk-2 protein
EKDSDSPSNKEKSICLKGKKKLLDFNALDPDEIIDPASLNLESDDSDEDADDGASENSYSSSDSSLRPYDLSDDDSDLKRKISQLADVVAALRKSNDADGVERAIDVAEKLIRASPDELKHAARDMTRTLVQVRCSDIALEGAEESTEDKRQRSLVALVVTCPFESLESLNN

SEQ ID NO: 28 - protein, 134 aa, unknown
  part of Glycine max clk-2 protein
MEEGLEKRELEGEVVTKVVEVVSAIKNAKHVDQVIRALHSLVTLLFPFDSSLLSDSIDQSYRDQVEVPSAEKRHAWWRAFYRGAAFPTLARFLLLDVASNWLGCFPFMAQKYIYDVFFVRGLVTDVLQILVPFL

SEQ ID NO: 29 - protein, 75 aa, unknown
  part of Glycine max clk-2 protein
EISTGLEWIRTWALDVAESDTDKECYTMAMTCIQLHVEMALQTSRALESVRNSLKAGPVLPSDASKVTIKIPHLN

SEQ ID NO: 30 - protein, 528 aa, unknown
  composite of Glycine max clk-2 protein
MEEGLEKRELEGEVVTKVVEVVSAIKNAKHVDQVIRALHSLVTLLFPFDSSLLSDSIDQSYRDQVEVPSAEKRHAWWRAFYRGAAFPTLARFLLLDVASNWLGCFPFMAQKYIYDVFFVRGLVTDVLQILVPFLEKDSDSPSNKEKSICLKGKKKLLDFNALDPDEIIDPASLNLESDDSDEDADDGASENSYSSSDSSLRPYDLSDDDSDLKRKISQLADVVAALRKSNDADGVERAIDVAEKLIRASPDELKHAARDMTRTLVQVRCSDIALEGAEESTEDKRQRSLVALVVTCPFESLESLNNEISTGLEWIRTWALDVAESDTDKECYTMAMTCIQLHVEMALQTSRALESVRNSLKAGPVLPSDASKVTIKIPHLNNWSNSYERELPPKPNQVKKGKTRRWSLQSPTQQNQMEYSHNKLPMYAAAFMLPAMEGYDKKRQGVDLLGRDFIVLGKLIYMLGVCMKSVAMHPEASMLAPSLLNMLRSREVCHHQEAYVRRAVLFAAACVLVALHPTYISSTLLEGN

SEQ ID NO: 31 - protein, 876 aa, unknown
  C. elegans clk-2 (QM37) mutant protein, with C to Y substitution at position 772
MNLRSRLVNATERAVLFQIFKDVQNDPEKYDNAVEAICESIDYFGKFLTDSEYLTQIKPILDTQCPTKSIICFSKCLTKVSTDINTTTFRDVITMLDWLKYVVEKSLTSAICSSLKVKETDVSAVQLYREFASACSNIPEKVSNCCAKALSGEHVKYINTVKWIFKMNLVQGIQKAMLLAHDDIVTAAPFTSFYGSGGPYMKTVAEIISSGRIDITNKDGLLVQMIEWIGSLNNFDSQWRRMMFLIFQEPTYQGIQVHESLLTTLFLISKSDQILKRCIEATDLTGTLKRVVMVKLPFQRVLKRKTIEILINFVYRTKEQFAIQLLETSVKIWSDLNYAKSAPESQERHIVRMILYLVHLFRTCSSIDWESLFLNSMDGVHCRMSMLPMYVQSGIFVNQALCKQATKHRSKTHGSDEQPPETLEENKFVSSEVGKIWFEEMTSILEHGFNSSTVKDSERVRETANEITKDDSGEEFEETNAQRLQNNKDSAAITSKNNLRLDSDDDEDFPDYQVNESEKIFKNLEIGEEPKNKVTPPAYIADAFEMLLEKEKYEVFEAAFFNITNLINRRPIGFPQIAEKLFIRILHLQNNFGTPKFKETVDEIAVACITQRPEIVPSVVRLIIAPGQGFSIKQRLLHYIHNAADGMGALDKKLEECVMAQQLRIGGPTLSIILHRTINTDYDDEDEDPHRLLVPEWRRMVDARIAANTRRIGTTREPPRAGVVNRLAQAAKYMFYPLLVLPRGENASLLGKDSDLLASLIMVASMVYVRYGVCPQIHRMSSELISYATPHRFSENAKLRTACIIAHLNVTTLLPGDLMDELFDVPALIGWFDWANSVLVNASSSQLEKDMTRQFGHSVTKHLQRHHPAVLQHQDV

SEQ ID NO: 32 - protein, 688 aa, unknown
  Tel2p, S. cerevisiae clk-2 protein
MVLETLKQGLDSSQIHEALIQLDSYPREPVDLDASMVLIKFVIPVYPSLPERSKVILRRLASKSFTFLCQIVTFSRTISGRDGLQEIRIYQEILEDIISFEPGCLTFYLKASTTSKADRDSIKALFFGSKLFNVLANRIDMAKYLGYLRLQWKFLLESNETDPPGFLGEWLVSSFLLNPVLAADMLLGELFLLKESYFFSFQKIISASSLIDQKRLIAKFLLPYIQVIVTLENLNDVRKILRRFDLDKIISLSVLFEIQSLPLKEVIVRLMSNHSSTKFVSALVSKFADFTDEEVDTKTCELLVLFAVHNLNHSQREEIAHDERFLNGVTKHLGSNEREARERAMFIAKLLSGGHLKYESDFKINIPNVKFESNSDDKIIDFQSLKNPSICNTQTDVGKDKITEVSGHVQSLTLDCSDSDDEDENDEREIVKRIVFLKDLMKEYEKTGESRKAPLIPLLKQTVKLIRQKADFQLEVGYYAQGILSSIVCLNNEFDEPLFEQWRINALTSILVVLPEKVNGAINILFNSELSLQQRMSLLSALGLSARELRGLDDPTIVKPKFDFPTNRLPWDDQSHHNSRLVEVQESTSMIKKTKTVWKSRKLGKDREKGTQNRFRKYAGLFFYPLAHGWLNGIDVGTYNQLFKSHYLTTLRIIYSCANPVHDFESMTELMNHIISSAIEEGISLNKG

SEQ ID NO: 33 - DNA, 1520 bp, C. elegans cex-7 cDNA
tcgaacttcaatttatatcgatgtttcttctattttagtgcaaattttaatcaaaaaaattaaaccttttgtcaacatggcgcgaaattttcccactgctctcgattacctgggatcagaagctgaagattttaacaaggcgcaacacttgtatctgaaaccgatggctgttataaaaattaccgtagtgctgcctcggatgacgatcccgggacaatcaatatctaattgggatctcatggaaagactaaaacgtgcaattgatccaattcaaatggattcttgcaaagttcgtgagagcaatatcgacagtgttatttttgaagcggaacttctttcgctaggaatcatgcagaaaacgatgaagattctcgatggattctctatgaaagtgtctggatttgctgagcccttgaaagttaaaacaaaagaggcaaagcttgattttccaagtcgtcatgattgggatgattggtttatgaaacataagatggacgagatgaaacccggagaacgtcctgatacagtttatttggcaagaattccagtgaaatggttctgcgatggttacaatgatcttccttctgaacgacgtcttcgcgttgcaatggaagcgttcggatctgttagagttgtcgacattccaatttgtgacccactccgatctcgaatgaattcaaaaatctctggtattcagcaaaaaggtttcggattagggcaggatgtattttttgaagcatatgtgcaatttatggagtataaaggtttcgccactgccatggattccctgagaaatcgtaaatgggcaaaacgaattgatggacggttctttcaggctaatgtcaaggtggatttcgatcgatcacgtcatctcagcgaagttcaaattgcaaagcgagcggaagaacgacgtcaaattgaaacggaacgactgcggcaagaagaagaggagttgaatatcagacgtcaagaagaactgaaagttaaacaagaactcgatgataaagatagaagacgagaagatagagaacggaaacgccgcgaaaagagggaactcgaacgaatggctgaagaagagaaaaaacgccttgaaaaggaacgacttgaagcagagcaacgatcacgagccactcgccgtttgcaaggtgttcgtcttttgaagtttctgtttgaaaaaattgaagcacgagaagagagacgaaagagaaaagaagaagaaaagttgaaagatgagctgagcaaaatcaaagaacttgaagaacaacctgtagaacaagaagacgcattgaggcaagctctacttcagcagagggaaattcgaatgcgagaacgcttgaaagagaagatgaaagcttcaggagcagagaaggataagaagagagataaaaataagacttctagaagaagacgaactaatagacatgacacttcatcctcttcatccgaagattctgattctccatctgattctccccactctcgtcgacaaagaaaacgtcaaagtgaaggagatgatttcc

SEQ ID NO: 34 - protein, 478 aa, unknown
  C. elegans cex-7 protein
MARNFPLYLGSEAEDFNKAQHLYLKPMAVIKITVVLPRMTIPGQSISNWDLMERLKRAIDPIQMDSCKVRESNIDSVIFEAELLSLGIMQKTMKILDGFSMKVSGFAEPLKVKTKEAKLDFPSRHDWDDWFMKHKMDEMKPGERPDTVYLARIPVKWFCDGYNDLPSERRLRVAMEAFGSVRVVDIPICDPLRSRMNSKISGIQQKGFGLGQDVFFEAYVQFMEYKGFATAMDSLRNRKWAKRIDGRFFQANVKVDFDRSRHLSEVQIAKRAEERRQIETERLRQEEEELNIRRQEELKVKQELDDKDRRREDRERKRREKRELERMAEEEKKRLEKERLEAEQRSRATRRLQGVRLLKFLFEKIEAREERRKRKEEEKLKDELSKIKELEEQPVEQEDALRQALLQQREIRMRERLKEKMKASGAEKDKKRDKNKTSRRRRTNRHDTSSSSSEDSDSPSDSPHSRRQRKRQSEGDDF

SEQ ID NO: 35 - protein, 550 aa, unknown [sequence omitted]

SEQ ID NO: 36 - DNA, 696 bp, C. elegans coq-4 cDNA
  clk
atgtctgctcaaaagctatacgcatcgcatgttcccttggcaccgctgtctcggatgcttctcgggattggatcagcagtaacagcgatctcggatccaaaaagaggagatatggtggcagcgatgggcgaaactactgcaattggaccagttttagaaaatattcgaaaaagaatggaatctgatgttgttggaaagcgacttcttctcgaaaaaccaagaatttcaaatggaacaattgatagaaagtggctaagacagttaccggatggaacattaggaaaattgtattcaaactttctcgatcgtttgaacacatctccagatgctcggcccactgtcaagtatatcgataatttggagcatctttatgttatgcaaaggtatcgcgaaacacacgacttcacccacatcgcattggagcagaaaacgaacatgctcggcgaggtaacagtcaaatatttcgaaggaattcaatatgggcttccaatgtgtgtcactggtggaatatttggaggtgccaggcttttaacaaaaaatcgccaagaacttgtcgaccggaacctcccttgggttgtggagcaggccacgaatgcacgattcttcatggctttcgactgggaaaatcactttgaaaagcagctcagcgaggtgcagaaggagctaaatataacgccattatctgtgaatatgtga

SEQ ID NO: 37 - protein, 263 aa, unknown
  C. elegans COQ-4 protein
MSAQKLYASHVPLAPLSRMLLGIGSAVTAISDPKRGDMVAAMGETTAIGPVLENIRKRMESDVVGKRLLLEKPRISNGTIDRKWLRQLPDGTLGKLYSNFLDRLNTSPDARPTVKYIDNLEHLYVMQRYRETHDFTHIALEQKTNMLGEVTVKYFEGIQYGLPMCVTGGIFGGARLLTKNRQELVDRNLPWVVEQATNARFFMAFDWENHFEKQLSEVQKELNITPLSELLDLPEMEPDVPDILFSKGHPGFWRVLQAVDMMI

SEQ ID NO: 38 - protein, 268 aa, D. melanogaster COQ-4
MMQRCLRLQKPLALRRGLRLAQANSQAVATEAPEAEPLDAFERQYLKERIEISPFQRLFLGAGSSIAALLNPRRHDMIACLGETTGEDALWTILDTMQASEEGQRIMADKPRIHTSTIDFKYLETLPPDTFGAAYVKFLKDNQVTPDSRMAVRFLEDPKLAYLMTRYRECHDLIHTVLDMPTNMLGEVAVKWVEALNTGLPMCYGGAVFGAVRLRPKQRRAYLKHYLPWALENGKRTKPLMPVYWEKRWEQNIHELRSELGITVLNKA

SEQ ID NO: 39 - protein, 265 aa, Homo Sapiens COQ-4
MATLLRPVLRRLCGLPGLQRPAAEMPLRARSDGAGPLYSHHLPTSPLQKALLAAGSAAMALYNPYRHDMVAVLGETTGHRTLKVLRDQMRRDPEGAQILQERPRISTSTLDLGKLQSLPEGSLGREYLRFLDVNRVSPDTRAPTRFVDDEELAYVIQRYREVHDMLHTLLGMPTNILGEIVVKWFEAVQTGLPMCILGAFFGPIRLGAQSLQVLVSELIPWAVQNGRRAHCVLNLYYERRWEQSLRALREELGITAPPMHVQGLA

SEQ ID NO: 40 - protein, 272 aa, S. cerevisiae COQ-4
MFYLNAHLEINKVVDVVMSLSKKFLKPSVASNQLRLLFTAAERKVNYPGHVPLSPLQRIFLVAGSAIMGLKAPWRGGDMISVLGDASGQPFFLHRLLNKMLVDKTGREILKDKPRMTSKSLNLPFLRTLPPNTLGKIYVDWIDKEHVGPDTRSPTRFVDDPEEAYVMQRYRESHDFYHAICNMPTNIEGELAIKWLEFVNMGLPVGALSALFGPLRLNCEQASRFRRVYIPWSIRNGLNAKTLINVYWEKELTNDIEDVRRRIRIEAAPPLV

SEQ ID NO: 41 - protein, 226 aa, Arabidopsis thaliana COQ-4
MIIERARVPLSRWQQAAVAMGSALGALVDPRRADLIAALGETTGKPAFEMVLERMKKSEEGRAILLERPRVVSEQVGHAWDLPENTFGAAYAKFMGSRNFSPDDRPPVRFMETDELAYVATRAREVHDLWHTLFGLPTNLIGESSLKVIEFEQMYLPMCMLSVIGGTVRFNEKQRSMFLKHYLPWAVRAGRQCTDLMCVYYERHFSEDLEQVRRKWGIIPAPQHPK

SEQ ID NO: 42 - protein, 167 aa, unknown
  part of Mus musculus COQ-4 protein
ALYNPYRHDMVAVLGETTGCHTLKFLRDQMKKDPEGAQILQERPRISLSTLDLSKLQSLPEGSLGREYLRFLDVNKVSPDTRAPTRFVDDEELAYVIQRYREVHDMLHTLLGMPTNMLGEVVVKWFEAVQTGLPMCILGALFGPIRLRTQSLQVLFSELIPWAIQNG

SEQ ID NO: 43 - protein, 123 aa, unknown
  part of Mus musculus COQ-4 protein
YPDHIPTTPLQKMLLAAGAAGMALYNPYRHDMVAVLGETTGCHTLKFLRDQMKKDPEGAQILQERPRISLSTLDLSKLQSLPEGSLGREYLRFLDVNKVSPDTRAPTRFVDDEXLAYVNQKYR

SEQ ID NO: 44 - protein, 89 aa, unknown
  part of Mus musculus COQ-4 protein
HLPTSYPSLSLGEVVVKWFEAVQTGLPMCILGALFGPIRLRTQSLQVLFSELIPWAIQNGRRATCVLNIYYEQRWEQPLTALREELGIS

SEQ ID NO: 45 - protein, 218 aa, Unknown
  Mus musculs consensus COQ-4 proteein
YPDHIPTTPLQKMLLAAGAAGMALYNPYRHDMVAVLGETTGCHTLKFLRDQMKKDPEGAQILQERPRISLSTLDLSKLQSLPEGSLGREYLRFLDVNKVSPDTRAPTRFVDDEELAYVIQRYREVHDMLHTLLGMPTNMLGEVVVKWFEAVQTGLPMCILGALFGPIRLRTQSLQVLFSELIPWAIQNGRRATCVLNIYYEQRWEQPLTALREELGIS

SEQ ID NO: 46 - protein, 137 aa, Glycine max COQ-4
LPANTFGAAYARFMGSRNFSPDDRPPVRFMDTDELAYVAMRAREVHDFWHTLFDLPTNLIGETALKVIEFEQMGLPMCLLSVIGGTARFSEKQRKLFYHHYFPWAIHAGMPSTDLMCVYYERHFDEDLEDVRRKLQI

SEQ ID NO: 47 - protein, 136 aa, Bos taurus COQ-4
YPEHIPTSVLQKVLLAAGSAGMALYDPYRHDMVAVLGETTGRRTLKVLRDQMKRDPEGAQILQERPRISLSTLDMGKLRSLPEGSFGCAYLHFLDVNRVSPDTRAPTRFVDDEELAYVIQRYREIHDMLHALLGMP

SEQ ID NO: 48 - protein, 120 aa, Medicago trunculata COQ-4
NFSPDDRPPVRFMDTDELAYVAMRAREVHDFWHTLFDLPTNLIGESALKVIEFEQMHLPMCVMSVLGGTARFSEKQRKLFYQHYFPWAVRAGTQCNDLMCVYYEQHFHQDLEDVRRKLGI

SEQ ID NO: 49 - protein, 113 aa, Ancylostoma caninum COQ-4
KFVQNSEHLYVMQRYRETHDFNHVLLQMPTHMLGEVTVKYFEGIQFGLPMCVTAGLFGAARLRKNHRHRFLTQHLPWIVEQATKGRFFMAIDWENHWEETIPSLQEQFGITPL

SEQ ID NO: 50 - protein, 113 aa, Trypanosoma cruzi COQ-4
  VARIANT (1)...(113): Xaa = Any Amino Acid
FVAATTRSIWDPVNADDVAAVGETPALTALGHMKQSMMSDRTGRMILRTQPRVTDETLEFASRQPPGTFGHRYAQFMKFXXFTPNGRTPVAHIADPTLAYVMQRQRETHDFLH

SEQ ID NO: 51 - protein, 99 aa, Rattus rattus COQ-4
HPYRHDMLPVLGETTGCHTLKFLRDQMKKDPEGAQILQERPRISLSTLDLSKLQSLPEGSLGREYLRFLNANKVSPDTRAPTRFVDDEELAYVIQRYRE

SEQ ID NO: 52 - protein, 122 aa, Gossypium hirsutum COQ-4
  VARIANT (1)...(122): Xaa = Any Amino Acid
IKLSPWQQAAVAVGSAVGALLDPRRADLIAALGETTGKPAFERVLERMRRSPEGKTXLLERPRVISANVGHAWDLPKNTFGAAYARFLGSXNFSPDDRPPVRFMDTDELAYVAMRAREVHDF

(19) United States
(12) Patent Application Publication
(10) Pub. No.: US 2003/0162291 A1
(43) Pub. Date: Aug. 28, 2003

(54) Clk-2, cex-7 and coq-4 genes, and uses thereof

(76) Inventors: Seigfried Hekimi, Montreal (CA); Claire Benard, Montreal (CA); Brenton McCright, Gaithersburg, MD (US); Bernard Lakowski, Paris (FR); Dong Han, Montreal (CA); Jean-Claude Labbe, Zurich (CH)

Correspondence Address: PENNIE AND EDMONDS, 1155 AVENUE OF THE AMERICAS, NEW YORK, NY, 100362711

(21) Appl. No.: 10/312,187

(22) PCT Filed: Jun. 20, 2001
(86) PCT No.: PCT/CA01/00913

(51) Int. Cl. A61K 48/00; C12N 5/02; C07H 21/04

(52) U.S. Cl. 435/375; 514/44; 536/23.2

(57) ABSTRACT

The present invention relates to a clk-2 gene which has a function at the level of cellular physiology involved in developmental rate, telomere length and longevity, wherein clk-2 mutations cause a longer life, an altered cellular metabolism and an altered telomere length relative to the wild type, wherein clk-2 overexpression leads to telomere shortening. The present invention also relates to clk-2 co-expressed gene which comprises a cex-7 gene having the nucleotide sequence set forth in FIG. 33 which codes for a CEX-7 protein having the amino acid sequence set forth in FIG. 34 wherein said gene is located in the clk-2 operon and said cex-7 gene is transcriptionally co-expressed with clk-2 gene present in said operon. The present invention also relates to a coq-4 gene which has a function at the level of cellular physiology involved in the regulation of developmental rate and longevity, wherein coq-4 mutations cause altered cellular metabolism and physiological relative to the wild type, wherein coq-4 gene has the identifying characteristics of nucleotide sequence set forth in FIG. 36.

### BACKGROUND OF THE INVENTION

(a) Field of the Invention

The invention relates to the identification of three genes: the gene clk-2, the gene cex-7 that is located in the same operon as clk-2, and the gene coq-4. The invention shows that these genes regulate the timing of development and behavior, and determine life span and that clk-2 regulates the length of telomeres.

(b) Description of Prior Art

A class of genes was identified in the nematode Caenorhabditis elegans, the clk (‘clock’) genes, whose activity controls how fast the worms live and die. Mutations in these genes result in an alteration of developmental and behavioral timing, including an average slow down of the animal's embryonic and post-embryonic development and of their rhythmic behaviors, as well as an increase in the animal's life span. In addition, mutations in these genes display a maternal effect, namely, homozygous mutants (clk/clk) derived from a heterozygous mother (clk/+), appear phenotypically wild-type.

We isolated the mutations that define the genes clk-1, clk-2, clk-3 in a screen for viable maternal-effect mutations in the nematode Caenorhabditis elegans (Hekimi, S. et al., Genetics 141, 1351 (1995)). gro-1 was originally identified by a spontaneous mutation isolated from a strain that had been recently established from a wild isolate (Hodgkin, J. and Doniach, T. Genetics 146, 149 (1997)). Our subsequent reappraisal of this mutation revealed that it shares the characteristics of the clk genes (Wong, A. et al., Genetics 139, 1247 (1995)).

We have molecularly identified two of these genes, clk-1 and gro-1. clk-1 encodes a protein that is highly conserved from proteobacteria to humans which is structurally similar to the yeast metabolic regulator Cat5p/Coq7p (Ewbank, J. J. et al, Science 275, 980 (1997); WO98/17823). gro-1 encodes a highly conserved cellular enzyme, the dimethylallyltransferase:tRNA dimethylallyltransferase (WO99/10482).

To date, clk-1 is the gene that has been characterized in greatest detail. In addition to the phenotypic and molecular characterization, it was found that clk-1 is ubiquitously expressed in the worm's body where it localizes to the mitochondria, the energy generating organelle of the cell (Felkai, S. et al, EMBO Journal 18, 1783 (1999)). clk-1 thus controls timing by regulating physiological rates (Branicky R, C. Benard, S. Hekimi, Bioessays 22, 48 (2000)).

The gene clk-2 is defined by one allele that was isolated in a screen for viable maternal-effect mutations in Caenorhabditis elegans (Hekimi, S. et al., Genetics 141, 1351 (1995)). The mutations in the gene clk-2 were shown to result in an alteration of the timing of several developmental and behavioral events (Hekimi, S. et al., Genetics 141, 1351 (1995)) and that the activity of the gene clk-2 controls how fast the worms live and how soon they die (Lakowski, B. and Hekimi, S. Science 272, 1010 (1996). We have also noticed other phenotypes of the clk-2 mutants such as the temperature sensitivity of the clk-2 (qm37) allele. Overall, these phenotypes are similar to those of mutations in the three clk genes (Hekimi, S. et al., Genetics 141, 1351 (1995)).

Results to date suggest that the effect of clk genes on the rate of aging is due to an effect on the rate of living. First, clk-1 mutations which lead to a decrease of clk-1 activity result in a slow down of development and behavior and in an increase in life span. On the other hand, overexpression of clk-1 in transgenic animals accelerates the rate of living as revealed by the absence of a characteristic behavioral slow down with age. Second, the effect of the different clk genes is additive. We have shown that double clk mutants develop more slowly and live longer than the single clk mutants. Third, clk genes are distinct from dauer formation genes (daf genes) which are involved in stress resistance and also prolong life span. Daf genes affect life span through a separate mechanism from that of clk. In fact, clk mutants are neither dauer constitutive nor dauer defective and daf-16 mutations cannot suppress the long life of clk-1, -2, -3 mutants.

The gene coq-4 is similar to the gene clk-1 in that both genes are required for normal ubiquinone biosynthesis in yeast and both genes have no homologues in E. coli. The gene cex-7 that will be described below has been found to be a pseudoautosomal gene named XE7 in humans.

It would be highly desirable to be provided with a detailed phenotypic and molecular characterization of the gene clk-2, as well as with a characterization of the gene coq-4 in an animal.

### SUMMARY OF THE INVENTION

One aim of the present invention is to provide with a clk-2 gene which has a function at the level of cellular physiology involved in developmental rate, telomere length and longevity, wherein clk-2 mutations cause a longer life, an altered cellular metabolism and an altered telomere length relative to the wild type, wherein clk-2 gene has the identifying characteristics of nucleotide sequence described in FIG. 1.

In accordance with the present invention there is provided the use of a clk-2 gene to alter a function at the level of cellular physiology involved in the regulation of developmental rates, telomere length and longevity, wherein clk-2 mutations cause a longer life, an altered cellular metabolism and physiological rates and an altered telomere length relative to the wild type, wherein clk-2 gene has the identifying characteristics of nucleotide sequences described in FIGS. 1, 4-7, 15, 16, and 20-24, or wherein the gene codes for a protein sequence as described in FIGS. 2, 3, 8-14, 17-19, and 25-32 as deduced from FIGS. 1, 4-7, 15, 16, 20-24.

Also is provided with the invention the use of a clk-2 gene to alter function at the level of cellular physiology involved in the regulation of developmental rate, telomere length and longevity, wherein clk-2 mutations cause a longer life and altered cellular metabolism and physiological rates and an altered telomere length relative to the wild type, wherein the gene codes for a protein having a sequence as set forth in FIG. 32.

In accordance with the invention there is provided a CLK-2 protein that has a function at the level of cellular physiology involved in the regulation of developmental rate, telomere length and longevity.

There is also provided with the invention a mutant CLK-2 protein which has the amino acid sequence described in FIG. 31, and the use of CLK-2 protein to alter a function at the level of cellular physiology involved in the regulation of developmental rates, telomere length and longevity, wherein the CLK-2 protein has the amino acid sequence as described in FIGS. 2, 3, 8-14, 17-19, and 25-32.

In accordance with the invention, there is provided a clk-2 gene which has the nucleotide sequence described in FIG. 1, and the use of clk-2 gene and homologues thereof, to manipulate the physiological rates and/or telomere biology, whereby life span of an organism is altered.

There is also provided a mouse which comprises a gene knockout of the murine clk-2 gene homologue to a clk-2 gene.

There is provided with the present invention a method to increase the life span of multicellular organism and metazoan which comprises altering the function of telomeres and mechanisms of sub-telomeric silencing.

The invention also provides the use of clk-2 gene, CLK-2 protein, and homologues thereof, for screening drugs which decrease or increase the life span of a multicellular organism, wherein the drug enhances or suppresses the expression of the clk-2 gene or activity of the protein CLK-2, and homologues thereof.

The use of a compound is provided with the invention for the manufacture of a medicament for increasing and/or decreasing physiological rates of tissues, organ, and/or whole organism of a host; wherein the compound is interfering with activity of CLK-2 protein and homologues thereof.

The use of a compound is also provided to promote tissue and/or organ specific reduction or increase of clk-2 activity for the manufacture of a medicament for the treatment of pathological conditions causing increase or decrease of physiological rate of tissue and/or organ in an individual, wherein the compound is interfering with activity of CLK-2 protein and homologues thereof.

In accordance with the invention there is provided a clk-2 co-expressed gene which comprises a cex-7 gene having the nucleotide sequence as described in FIG. 33, and which codes for a CEX-7 protein having the amino acid sequence described in FIG. 34 wherein the gene is located in the clk-2 operon and the cex-7 gene is transcriptionally co-expressed with clk-2 gene present in the same operon.

A human homologue of cex-7 gene is also provided with the invention, wherein the gene codes for a protein having a sequence as described in FIG. 35.

There is provided with the invention the use of a human homologue of cex-7 gene and homologues thereof to alter a function at the level of cellular level physiology involved in the regulation of developmental rates and longevity wherein the gene codes for a protein having a sequence as described in FIG. 35.

The invention provides with a mouse which comprises a gene knock out of the murine cex-7 gene homologue of the human gene described in FIG. 35.

There is also provided with the invention the use of a compound for the manufacture of a medicament for increasing and/or decreasing physiological rates of tissues, organ, and/or whole organism of a host; wherein the compound is interfering with activity of CEX-7 and homologues thereof.

Another aim of the invention is to provide with the use of a compound which promotes tissue and/or organ specific reduction or increase of cex-7 activity for the manufacture of a medicament for the treatment of pathological conditions causing increase or decrease of physiological rate of tissue and/or organ in an individual, wherein the compound is interfering with activity of CEX-7 and homologues thereof.

There is provided with the invention a coq-4 gene which has a function at the level of cellular physiology involved in the regulation of developmental rate and longevity, wherein coq-4 mutations cause altered cellular metabolism and physiological relative to the wild type, wherein coq-4 gene has the identifying characteristics of nucleotide sequence as described in FIG. 36.

A coq-4 gene provided with the invention has a function at the level of cellular physiology involved in the regulation of developmental rate and longevity, wherein coq-4 mutations cause altered cellular metabolism and physiological relative to the wild type, wherein coq-4 gene has the identifying characteristics of nucleotide sequence as described in FIG. 36, and the gene codes for a protein having a sequence as described in FIG. 37.

In accordance with the invention, there is provided with the use of coq-4 gene to alter a function at the level of cellular physiology involved in the regulation of developmental rates, wherein coq-4 mutations cause an altered cellular metabolism and physiological rates relative to the wild type, wherein the gene codes for a protein having a sequence as described in FIGS. 43 to 54 and homologues thereof.

There is also provided with the invention a mouse which comprises a gene knock out of the murine coq-4 gene as described in FIG. 47.

There is provided also with the invention the use of a compound for the manufacture of a medicament for increasing and/or decreasing physiological rates of tissues, organs and/or whole organism of a host; wherein the compound is interfering with activity of COQ-4 and homologues thereof.

A compound in accordance with the invention is provided to promote tissue and/or organ specific reduction or increase of coq-4 activity for the manufacture of a medicament for the treatment of pathological conditions causing increase or decrease of physiological rate of tissue and/or organ in an individual, wherein the compound is interfering with activity of COQ-4 and homologues thereof.

Having the clk genes in hand can serve to manipulate the rate of development, the cell cycle, the rate of behavior and the rate of aging. Another way to look at it is that it can help to control physiological rates including for medical and industrial purposes. Slowing down the rate of aging of individual organs or tissues to slow down their rate of deterioration is one medical example; accelerating the growth of farm animals or crops is an example of industrial utilization.

Here we describe our analysis of clk-2 and cex-7 and the inventions that result from this analysis, including the molecular characterization of clk-2 and cex-7 and the identification of homologues in several species, including humans. We also describe our identification of a new clk gene: coq-4. We have obtained a mutation in the worm coq-4 locus and have shown that the mutant animals display several of the most important characteristics of clk mutations.

### BRIEF DESCRIPTION OF THE DRAWINGS

FIGS. 1A and 1B illustrate the Caenorhabditis elegans clk-2 cDNA sequence.

FIG. 2 illustrates the Caenorhabditis elegans CLK-2 protein sequence.

FIG. 3 illustrates the Homo sapiens CLK-2 protein sequence(derived from clone KIAA0683).

FIGS. 4A and 4B illustrate the Homo sapiens clk-2 homologue nucleotide sequence (derived from AL080126).

FIG. 5 illustrates part of Mus musculus clk-2 cDNA sequence (derived from AA671905 vl11b10.r1).

FIG. 6 illustrates part of Mus musculus clk-2 cDNA sequence (derived from AA031108 mi40f03.r1).

FIG. 7 illustrates part of Mus musculus clk-2 cDNA sequence (derived from AA230994 mw30h11.r1).

FIG. 8 illustrates part of Mus musculus CLK-2 protein sequence (derived from gb|AA671905.1|AA671905.)

FIG. 9 illustrates part of Mus musculus CLK-2 protein sequence (derived from gb|AA230994.1|AA230994).

FIG. 10 illustrates part of Mus musculus CLK-2 protein sequence (derived from gb|AA031108.1|AA031108).

FIG. 11 illustrates Mus musculus composite CLK-2 protein sequence.

FIG. 12 illustrates part of Sus scrofa CLK-2 protein sequence (derived from gb|AW429611.1|AW429611).

FIG. 13 illustrates the Drosophila melanogaster CLK-2 protein sequence.

FIG. 14 illustrates the putative Arabidopsis thaliana CLK-2 protein sequence (derived from 7630034|emb|CAB88328.1|).

FIG. 15 illustrates part of Oryza sativa clk-2 cDNA sequence (derived from AU031811).

FIG. 16 illustrates part of Oryza sativa clk-2 cDNA sequence (derived from D24238).

FIG. 17 illustrates part of Oryza sativa CLK-2 protein sequence (derived from dbj|D24422.1|D24422).

FIG. 18 illustrates part of Oryza sativa CLK-2 protein sequence (derived from dbj|AU031811.1|AU031811).

FIG. 19 illustrates Oryza sativa composite CLK-2 protein.

FIG. 20 illustrates part of Glycine max clk-2 cDNA sequence (derived from AI461201 sa76d07.y1 Gm-c1004).

FIG. 21 illustrates part of Glycine max clk-2 cDNA sequence (derived from AW185029 se85g06.y1 Gm-c1023).

FIG. 22 illustrates part of Glycine max clk-2 cDNA sequence (derived from AW350166 GM210007A10F4R Gm-r1021).

FIG. 23 illustrates part of Glycine max clk-2 cDNA sequence (derived from AW397826 sg68g12.y1 Gm-c1007).

FIG. 24 illustrates part of Glycine max clk-2 cDNA sequence (derived from AW567713 si54a01.y1 Gm-r1030).

FIG. 25 illustrates part of Glycine max CLK-2 protein sequence (derived from gb|AW350166.1|AW350166).

FIG. 26 illustrates part of Glycine max CLK-2 protein sequence (derived from gb|AI461201.1|AI461201).

FIG. 27 illustrates part of Glycine max CLK-2 protein sequence (derived from gb|AW|85029.1|AW185029).

FIG. 28 illustrates part of Glycine max CLK-2 protein sequence (derived from gb|AW567713.1|AW567713).

FIG. 29 illustrates part of Glycine max CLK-2 protein sequence (derived from gb|AW397826.1|AW397826).

FIG. 30 illustrates Glycine max CLK-2 composite protein sequence.

FIG. 31 illustrates the Caenorhabditis elegans CLK-2 (QM37) mutant protein, with C to Y substitution at position 772.

FIG. 32 illustrates Tel2p, the Saccharomyces cerevisiae CLK-2 protein.

FIG. 33 illustrates the Caenorhabditis elegans cex-7 cDNA sequence.

FIG. 34 illustrates the Caenorhabditis elegans CEX-7 protein sequence.

FIG. 35 illustrates the Homo sapiens CEX-7 protein sequence (XE7).

FIG. 36 illustrates the Caenorhabditis elegans coq-4 cDNA sequence.

FIG. 37 illustrates the Caenorhabditis elegans COQ-4 protein sequence.

FIGS. 38A and 38B illustrate the comparison of CLK-2 eukaryotic homologues (hCLK-2: Homo sapiens CLK-2: Caenorhabditis elegans Tel2p: Saccharomyces cerevisiae AtCLK-2: Arabidopsis thaliana).

FIG. 39 illustrates the comparison of CLK-2 animal homologues (D.m.: Drosophila melanogaster, H.s.: Homo sapiens C.e.: Caenorhabditis elegans).

FIG. 40 illustrates the comparison of CLK-2 vertebrate homologues (H.s.: Homo sapiens, M.m.: Mus musculus, S.s.: Sus scrofa).

FIG. 41 illustrates the comparison of CLK-2 plant homologues (A.t.: Arabidopsis thaliana, G.m.: Glycine max, O.s.: Oryza sativa).

FIG. 42 illustrates the comparison of COQ-4 homologous proteins.

FIG. 43 illustrates Drosophila melanogaster COQ-4 protein (derived from gi|7293987|gb|AAF49344.1|CG3877).

FIG. 44 illustrates Homo sapiens COQ-4 protein (derived from gi|7705807|ref|NP_{—}057119.1|CGI-92).

FIG. 45 illustrates Schizosaccharomyces pombe COQ-4 protein (derived from gi|7493130|pir||T37755).

FIG. 46 illustrates Arabidopsis thaliana COQ-4 protein (derived from gi|4406761|gb|AAD20072.1|).

FIG. 47A illustrates part of Mus musculus COQ-4 protein (derived from gb|AA274683.1|AA274683); FIG. 47B part of Mus musculus COQ-4 protein (derived from dbj|AU051632.1|AU051632); FIG. 47C part of Mus musculus COQ-4 protein (derived from gb|AI157531.1|AI157531); and FIG. 47D Mus musculus COQ-4 consensus protein.

FIG. 48 illustrates Glycine max COQ-4 protein (derived from gb|AW201157.1|AW201157).

FIG. 49 illustrates Bos taurus COQ-4 protein (derived from gb|AW660771.1|AW660771).

FIG. 50 illustrates Medicago truncatula COQ-4 protein (derived from gb|AW696025.1|AW696025).

FIG. 51 illustrates Ancylostoma caninum COQ-4 protein (derived from gb|AW870537.1|AW870537).

FIG. 52 illustrates Trypanosoma cruzi COQ-4 Protein (derived from gb|AW330043.1|AW330043).

FIG. 53 illustrates Rattus rattus COQ-4 protein (derived from gb|AA800046.1|AA800046).

FIG. 54 illustrates Gossypium hirsutum COQ-4 protein (derived from gb|A|731097.1|AI731097).

FIGS. 55A-C illustrate the expression pattern of clk-2.

FIGS. 56A-E illustrate the telomere-lengthening phenotype of clk-2(qm37) mutants at different temperatures.

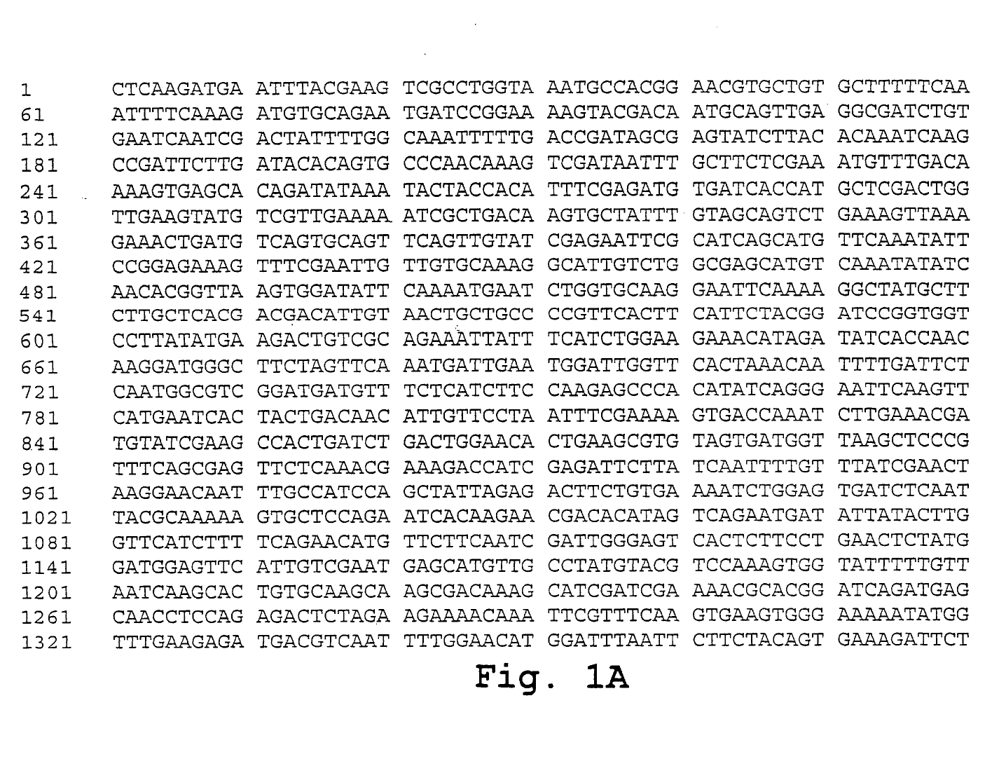

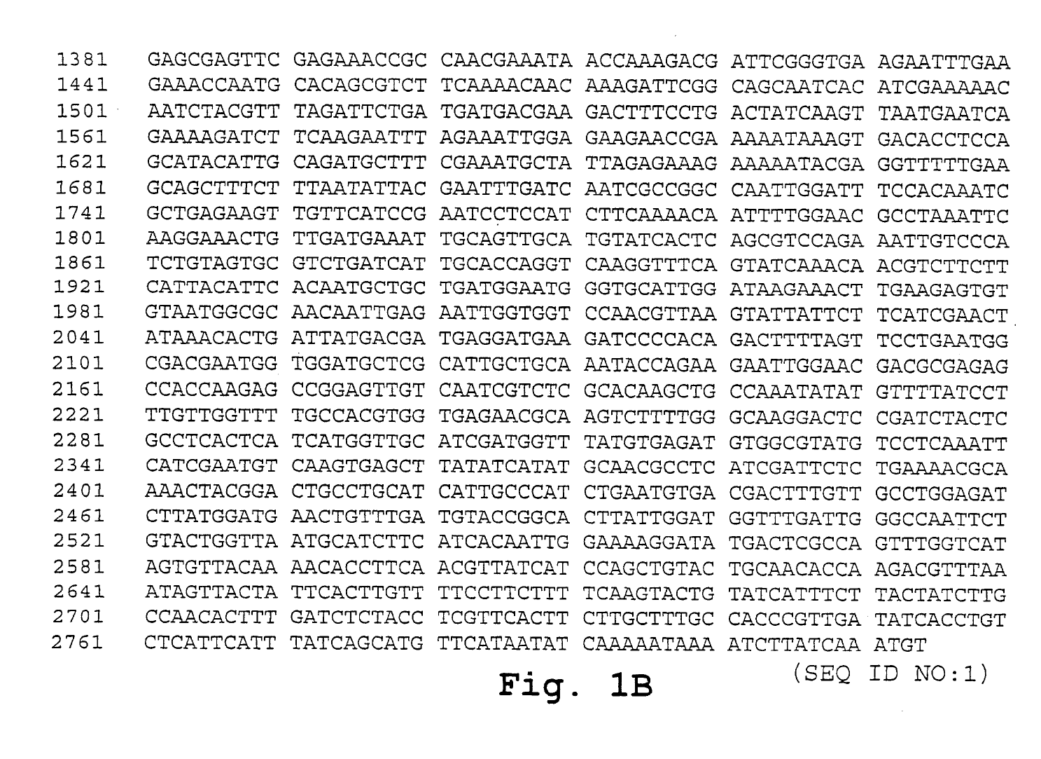

### DETAILED DESCRIPTION OF THE INVENTION

For the first time, there is provided with the present invention a new method of increasing life span by modulating the biology of telomeres.

The Clk Phenotype of clk-2 Mutants

We had shown previously that clk-2 mutants have a phenotype similar to that of clk-1 mutants, including the maternal rescue effect, their slow development and behavior, and their increased life span (Hekimi, et al., Genetics 141, 1351 (1995); Lakowski, B. and Hekimi, S. Science 272, 1010 (1996). We have characterized the defects of clk-2 mutants in much further detail, the results of which follow. From 15° C. to 20° C. the phenotype of clk-2 mutants is similar to that of clk-1 mutants. The average developmental, reproductive and behavioral rates are dramatically slower, and the mean and maximum life span longer, than those of the wild type as summarized in Table 1. In particular, the embryonic development of clk-2(qm37) mutants lasts 17.0±1.5 hours (n=97) at 20° C., while the wild type lasts 13.2±0.7 hours (n=80). The post-embryonic development of clk-2 (qm37) mutants is also slower lasting 95.7±1.3 hours at 20° C. (n=73), while the wild-type worms take only 53.6±8.7 hours (n=184).

The defecation cycles are slowed down as well, occurring every 105.7±15.2 seconds in clk-2 mutants at 20° C. (n=10) and every 54.9±0.6 seconds in the wild type (n=70). The pumping rate is lower, 180.9±24.8 pumps per minute occurring in the clk-2 mutants at 20° C. (n=25), and 265.3±64.4 pumps per minute in the wild type (n=25).

TABLE 1
Phenotypic characterization of clk-2(qm37)
animals at 20° C.

| | | | Maternally |
|---|---|---|---|
| | Wild Type | | rescue clk- |
| | (N2) | clk-2(qm37) | 2(qm37) |
| Embryonic | 13.2 ± 0.7 | 17.0 ± 1.5 | 13.3 ± 1.6 |
| Development | n = 80 | n = 97 | n = 40 |
| (hours) | | | |
| Post-embryonic | 53.6 ± 8.7 | 95.7 ± 1.3 | 53.9 ± 12.4 |
| Development | n = 184 | n = 73 | n = 98 |
| (hours) | | | |
| Self-brood | 302.4 ± 30.5 | 83.4 | 113.9 ± 30.3 |
| Size | n = 20 | n = 10 | n = 24 |
| (eggs) | | | |
| Peak Egg- | 5.3 | 1.3 | 3.6 ± 0.9 |
| laying Rate | n = 10 | n = 10 | n = 24 |
| (eggs per | | | |
| hour) | | | |
| Defecation | 54.9 ± 0.6 | 105.7 ± 15.2 | 60.3 ± 9.0 |
| (seconds) | n = 70 | n = 10 | n = 8 |
| Pumping | 265.3 ± 64.4 | 180.9 ± 24.8 | 245.2 ± 24.6 |
| (pumps per | n = 25 | n = 25 | n = 11 |
| minute) | | | |

In addition, we have also examined the self-brood size at 20° C. and found that is reduced in clk-2 mutants where it is 83.4 (n=10), while it is 302.4±30.5 in the wild type (n=20). The peak egg-laying rate is 1.3 (n=10) in clk-2 mutants at 20° C., and 5.3 (n=10) in the wild type. We have also examined the life span. clk-2(qm37) mutants live longer than the wild type, living on average 22.4±7.4 days (n=100) at 20° C. and having a maximum life span of 40 days, which is longer that the average life span of 19.3±5.3 days (n=100) and maximum life span of 32 days of wild-type N2 worms.

The developmental and behavioral phenotypes are fully maternally rescued, that is to say that homozygous clk-2/clk-2 mutants derived from a clk-2(qm37)/+heterozygous mother display wild-type phenotypes. In fact, the embryonic development of homozygous mutants derived from a heterozygous mother takes only 13.3±1.6 hours (n=40) and their post-embryonic development lasts only 53.9±12.4 hours (n=98) at 20° C. Also maternally rescued are both defecation, which occurs every 60.3±9.1 seconds at 20° C. (n=8) and pumping, which occurs at a rate of 245.2±24.6 pumps per minute at 20 C. (n=11). However, the reproductive phenotypes are only partially rescued by a wild-type copy of the gene clk-2 in the mother. The self-brood size is 113.9±30.3 at 20° C. (n=24), and the peak egg-laying rate is 3.6±0.9 (n=24). This indicates that the wild-type clk-2 gene in the mother induces an epigenetic state that lasts for only one generation. Erasure of the epigenetic state in the germ-line prevents the animal from having a wild-type rate of reproduction. In addition, the life span of maternally rescued homozygous mutants is dramatically shortened vs. both the mutant and the wild-type life span. Indeed, homozygous mutants derived from a heterozygous mother live only 14.9±4.1 days on average (n=106) and have a maximum life span of 27 days at 20° C. Interestingly, wild-type siblings of maternally rescued clk-2 live slightly shorter than wild-type N2 worms, 17.3±4.1 days (n=206). This observation indicates that wild-type physiological rates imposed by a maternal epigenetic setting are deleterious to animals that are partially incapable of regulating their physiological rates in response to environmental conditions.

TABLE 2
Life span of mutants and double mutant
combinations at 20° C. indicated in days

| | | Maximum Life |
|---|---|---|
| Genotype | Mean Life Span | Span |
| Wild type (N2) | 19.3 ± 5.3 | 32 |
| | n = 100 | |
| clk-2(qm37) | 22.4 ± 7.4 | 40 |
| | n = 100 | |
| Maternally | 14.9 ± 4.1 | 27 |
| rescued | n = 106 | |
| clk-2 (qm37) | | |
| Wild type (N2) | 18.4 ± 4.6 | 31 |
| | n = 260 | |
| clk-2(qm37) | 22.9 ± 7.3 | 45 |
| | n = 260 | |
| daf-16(m26) | 18.1 ± 2.6 | 25 |
| | n = 260 | |
| daf-16(m26) clk- | 21.7 ± 5.8 | 41 |
| 2(qm37) | n = 260 | |
| daf-2 (e1370) | 29.3 ± 10.3 | 51 |
| | n = 50 | |
| daf-2(e1370) clk- | 54.5 ± 21.4 | 101 |
| 2(qm37) | n = 50 | |
| eat-2 (ad465) | 30.0 ± 7.0 | 42 |
| | n = 34 | |
| eat-2(ad465) clk- | 26.6 ± 6.3 | 45 |
| 2(qm37) | n = 50 | |

We characterized the life span increase produced by clk-2 (qm37) by comparing it to that produced by other aging genes as summarized in table 2. Among the other genes that affect life span in worms, the best understood are the daf genes. Mutations in the eat genes prolong life span through caloric restriction by reducing the food intake of the animals, a process that also prolongs life span in vertebrates. Mutations in daf genes prolong life span by partial activation of the dauer formation pathway. The dauer stage is a dormant, long-lived, alternative developmental stage which is induced by adverse environmental conditions. The increased life span of all dauer formation mutants that have been tested is suppressed by loss of function mutations in daf-16.

In fact, we found that while daf-16(m26) lives 18.1±2.6 days on average with a maximum life span of 25 days, the double mutants daf-16(m26) clk-2(qm37) lives an average life span of 21.7±5.8 days with a maximum life span of 41 days. Furthermore, although double mutants with two long-lived dauer formation mutations do not live longer than mutants carrying only one of the component mutations, daf-2(e1370) clk-2(qm37) double mutants live substantially longer than daf-2, almost three times longer than the wild type. We have shown that while daf-2(e1370) lives 29.3±10.3 days on average with a maximum life span of 51 days, the double mutants daf-2(e1370) clk-2(qm37) lives an average life span of 54.5±21.4 days with a maximum life span of 101 days. In contrast to these observations, the effects of clk-2 and eat-2 are not additive. In fact, the double mutants live somewhat shorter than eat-2 mutants. We have shown that eat-2(ad465) lives 30.0±7.0 days on average with a maximum life span of 42 days, and that the double mutants daf-2(e1370) clk-2(qm37) live 26.6±6.3 days on average with a maximum life span of 45 days. These observations are also consistent with the finding that daf-2 eat-2 double mutants live longer than daf-2 or eat-2 mutants in isolation (Lakowski, B. and Hekimi, S. Science 272, 1010 (1996)). Together, these results show that daf-2 and clk-2 prolong life span by distinct mechanisms but that clk-2 works in a way that resembles caloric restriction.

The Strict Maternal Effect of the clk-2(qm37) Mutation

In addition to the Clk phenotype displayed by clk-2(qm37) mutants, they exhibit a temperature-sensitive embryonic lethal and sterile phenotypes at 25° C. We knew that qm37 is a temperature sensitive mutation and that the mutants lay dead embryos when they are transferred to 25° C. (Hekimi, S. et al., Genetics 141, 1351 (1995)). These findings have now been extended, and the phenotype of clk-2 mutants at 25° C. has been examined after a number of temperature shift experiments at different stages of development, from permissive to restrictive temperature and vice versa.

At the permissive temperatures (15 to 20° C.), clk-2 embryos all develop normally and grow up to become long-lived adults. However, when hermaphrodites that have developed at a permissive temperature are transferred to 25° C. before egg-laying begins, they produce only progeny that dies during embryogenesis at various stages of development. When these hermaphrodites, that have been producing dead embryos at 25° C., are transferred back to 18° C., they lay only dead eggs at first, but start to lay live eggs that develop into adults after having been 5-6 hours at 18° C. When hermaphrodites that are kept at 18° C., and that lay only live eggs, are transferred to 25° C. it also takes 5-6 hours before they lay only dead eggs. Both conditions (laying live or dead progeny) are fully reversible upon temperature shift even when the animal's entire post-embryonic development was carried out at a single temperature (permissive or non-permissive). In addition, when larvae that developed at the permissive temperature are shifted to 25° C., some arrest development and others reach a sterile and sick adulthood. These phenotypes are fully reversible as well. Finally, all these lethality and sterility phenotypes displayed by clk-2(qm37) mutants at 25° C. can be fully maternally rescued: heterozygous animals produce only live progeny at any temperature.

We have also found that the embryonic lethality at 25° C. is a strict maternal phenotype. That is to say that despite qm37 behaving as a recessive mutation, a wild-type allele in the genome of the embryo is not sufficient for survival if the mother was clk-2/clk-2 homozygous mutant. When clk-2 hermaphrodites are mated to wild-type males at 25° C. they nonetheless produce only dead embryos. When shifted to 18° C. at various times after mating they produce live males, indicating that the mating was successful. The strictly maternal lethal action of clk-2 indicates a very early focus of action, before activation of the zygotic genome.

To establish how early clk-2 acts during the development of the worm, we dissected embryos at the 2-4 cell stage from wild-type N2 and clk-2 mutant hermaphrodites kept at either permissive (20° C.) or non-permissive (25° C.) temperature and transferred them to the other temperature (or not, as a control). As summarized in table 3, we found that when development up to the 2-4 cell stage proceeded at the permissive temperature, almost all eggs hatched and carried out further embryonic and post-embryonic development at 20° C. {100% of dissected N2 eggs (n=35) hatched and 87% of dissected clk-2 eggs hatched (n=91)} or 25° C. {97% of dissected N2 eggs (n=36) hatched and 91% of dissected clk-2 eggs hatched (n=93)}. In contrast, when eggs had carried out development up to the 2-4 cell stage at 25° C. and were then transferred to 20° C., only very few clk-2 eggs hatched and succeeded in completing development at 20° C. {12% of dissected clk-2 eggs hatched (n=136)}. As a control, when N2 eggs had carried out development up to the 2-4 cell stage at 25° C. and were then transferred to 20° C., almost all hatched and succeeded in completing development at 20° C. {98%, n=45}, or at 25° C. {96%, n=45}. These results indicate that clk-2 is required for viability before the 2-4 cell stage. clk-2 is required in a narrow window between the very end of oogenesis and the initiation of embryonic development.

TABLE 3
Survival of eggs at the 2-4 cell stage, dissected from
mothers raised at 20 or 25° C. and transferred or not to
another temperature

| | % of eggs that | % of eggs that |
|---|---|---|
| | hatch when | hatch when |
| | developing at | developing at |
| Mothers | 20° C. | 25° C. |
| N2 at 20° C. | 100 | 97 |
| | n = 35 | n = 36 |
| clk-2 at 20° C. | 87 | 91* |
| | n = 91 | n = 93 |
| N2 at 25° C. | 98 | 96 |
| | n = 45 | n = 45 |
| clk-2 at 25° C. | 12* | n.d. |
| | n = 136 | |

Eggs that have reached the 2-4 cell stage at 20° C. are viable even when further development is carried out at 25° C., while eggs that reached the 2-4 cell stage at 25° C. die even when developing subsequently at 20° C.
*Only hatching is recorded in this table, but it should be noted that the 12% of clk-2 eggs transferred from 25 to 20° C. that succeed in hatching do not subsequently complete post-embryonic development, while the majority of the 91% clk-2 eggs that hatch when transferred from 20 to 25° C. reach adulthood.

Indeed, clk-2 hermaphrodites that have spent 26 hours of adulthood at 25° C., carry on average 9.9 developing eggs in the uterus (n=125), but produce on average 10.7 dead eggs (n=133) when shifted down to permissive temperature. This observation indicates that, upon transfer from the lethal temperature, only one oocyte or embryo dies on average in addition to those that have already formed an eggshell. This corresponds to the time at which fertilization, oocyte meiosis, pronuclear formation and eggshell formation occurs. We observed early embryonic development using DIC microscopy but did not detect any obvious abnormality in the events which follow fertilization. The early embryos look invariably normal and healthy with cells and nuclei of normal size and shape. We also visualized DNA using Dapi in oocyte and early embryos and did not detect abnormal patterns of chromosome segregation or any other defects. Finally, meiosis per se is not affected as clk-2 homozygous males can sire abundant cross-progeny at 25° C. when mated to wild-type hermaphrodites.

clk-2 Positional Cloning, Gene Structure and Operon

We have molecularly identified the gene clk-2 by positional cloning. The gene was localized on the genetic map within an interval of 0.84 cM on the left cluster of linkage group III of Caenorhabditis elegans, between the genetic markers sma-4 and mab-5 (Hekimi, S. et al., Genetics 141, 1351 (1995)). We refined this genetic position by a series of additional mapping experiments involving the genetic markers sma-3, unc-36, lin-13, and lin-39 by multi- and two-point crosses. The following multi-point results were obtained (the genotypes whose progeny was scored is given in brackets): dpy-17 14 clk-2 18 unc-32 (clk-2/dpy-17 unc-32); lon-1 47 clk-2 23 unc-36 (clk-2/lon-1 unc-36); sma-4 35 clk-2 3 mab-5 14 unc-36 (clk-2/sma-4 mab-5 unc-36); sma-3 18 clk-2 0 lin-13 10 unc-36 (sma-3 clk-2 unc-36/lin-13); clk-2 3 lin-13 49 unc-32 (lin-13/clk-2 unc-32); sma-3 40 lin-39 0 clk-2 33 unc-36 (sma-3 clk-2 unc-36/lin-39). In addition, a two-point cross was carried out (clk-2 unc-36/++) and 5/630 Uncs were found to develop quickly (p=0.4 cM). We also found that the deletion nDf2O does not delete clk-2 and that the duplication qDp3 does include clk-2. We thus placed the gene clk-2 within an interval of 0.3 cM, between sma-3 (at −0.9 cM on LGIII) and lin-13 (at −0.6 cM on LGIII), and lying very close to the gene lin-39 (at −0.65 cM).

By aligning the genetic and physical maps, we predicted the physical region which likely would contain the clk-2 gene. Groups of cosmids from this region were tested for their ability to rescue the clk-2 mutant by DNA microinjection. clk-2 was rescued by a pool of 4 cosmids (H14A12, K07D8, C34A5, C07H6). Individual injection of cosmids C07H6 and C34A5 also rescued the clk-2 phenotype, narrowing the physical position of clk-2 to within approximately 15 kb. Fragments of cosmid C07H6 (obtained by restriction digests from base pair 31,528 to base pair 36,545 of cosmid C07H6 [Accession: AC006605]) were then tested for rescue and a short region of approximately 5 kb was shown to fully rescue the phenotype, indicating that this 5 kb fragment contains the clk-2 gene.

The identity of the gene was further confirmed by phenocopying the clk-2 phenotype with RNA interference (RNAi) experiments, that is the injection of double stranded RNA corresponding to the coding mRNA sequence of a gene of interest to fully abolish the function of this gene. Double stranded RNA was produced by in vitro transcription from a cDNA (EST 447b4, gift of Y. Kohara) that mapped to this region, and injected into wild-type as well as into clk-2(qm37) worms. All wild-type and clk-2 animals injected with clk-2 dsRNA initially produced embryos that hatched and developed into worms phenotypically resembling clk-2(qm37), that is, slow development, slow defecation and sterility. After 24 hours, the injected animals started laying only dead eggs. These results confirmed the identity of clk-2. The observation that RNAi-treated mothers produce dead eggs, a phenotype more severe than the weak embryonic lethality normally present in the clk-2(qm37) strain, indicated that qm37 is a partial loss-of-function mutation that displays the null phenotype only at 25° C. We further confirmed the identity of the gene by characterizing the molecular lesion underlying the clk-2 mutation. Genomic DNA from the clk-2(qm37) strain was isolated and the nucleotide sequence of the clk-2 region determined. The qm37 mutation is a G->A transition at in base 2321 of the cDNA.

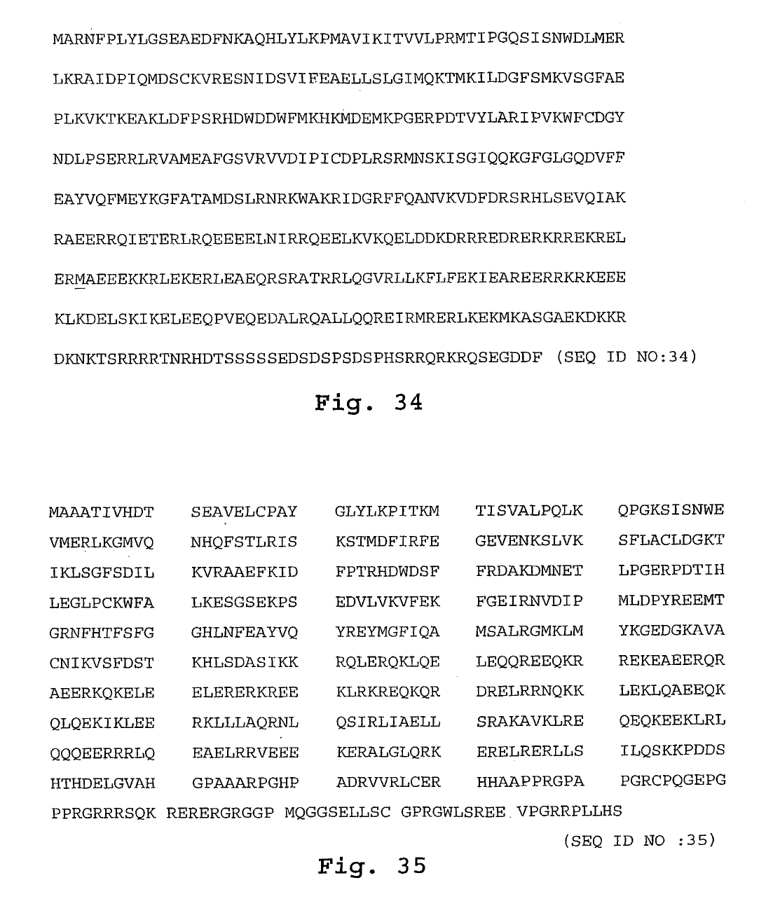

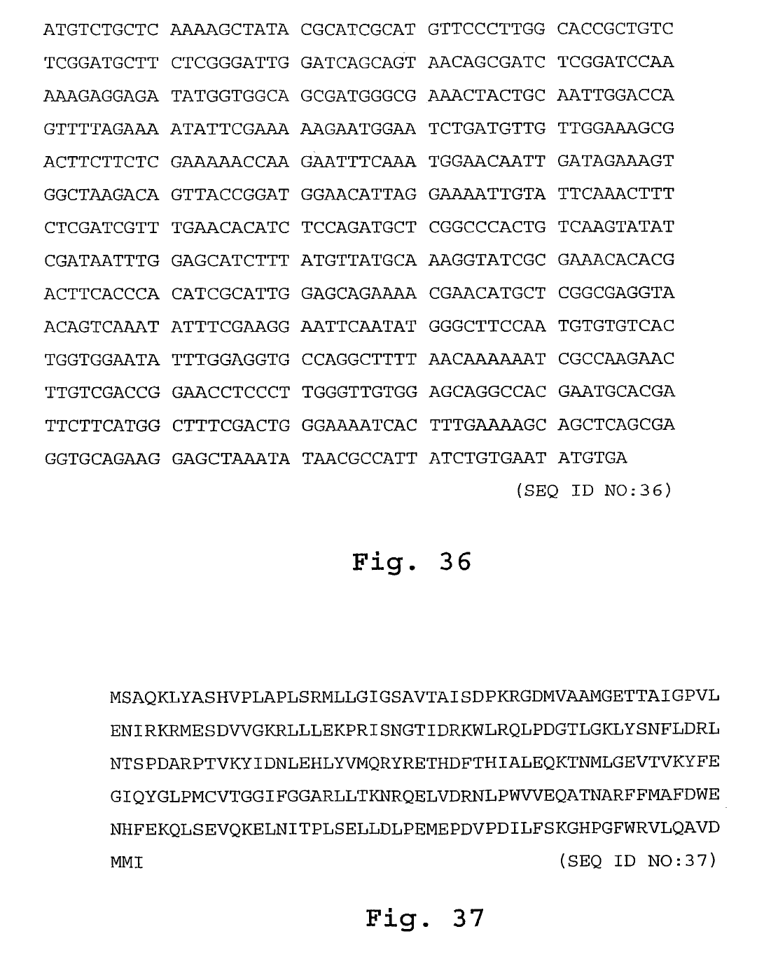

The structure of the gene was established experimentally by determining the nucleotide sequence of the EST yk447b4 cDNA, thus defining the actual intron/exon boundaries in vivo and allowing to predict the encoded protein. The gene clk-2 is SL2 transpliced. We have further established the gene structure by RT-PCR experiments, which not only showed that clk-2 is SL2 transpliced, but also that the gene just upstream to clk-2, which we called cex-7, is expressed and is SL1 transpliced. The transplicing by SL1 of a gene placed upstream, and by SL2 of a gene downstream constitutes a hallmark of genes which are in an operon, and are transcriptionally co-expressed. Therefore, clk-2 and cex-7 are transcriptionally co-expressed, and thus play functionally related roles. The cDNA (yk215f6) that corresponds to cex-7 was also sequenced. The gene cex-7 encodes a predicted protein of 481 amino acid residues in length (FIG. 34), that is similar to a human polypeptide of 550 amino acids (FIG. 35).

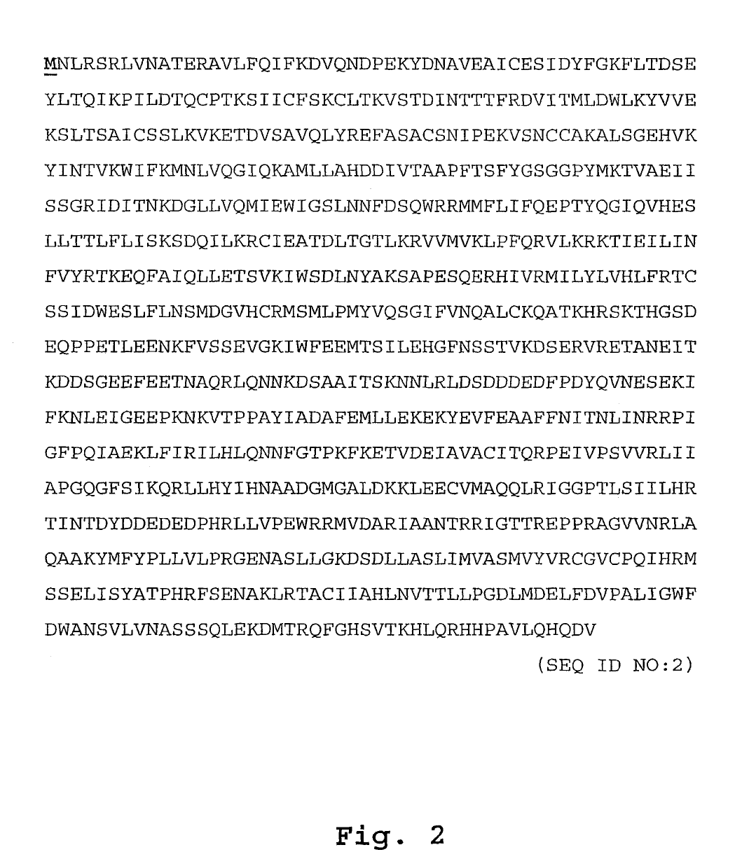

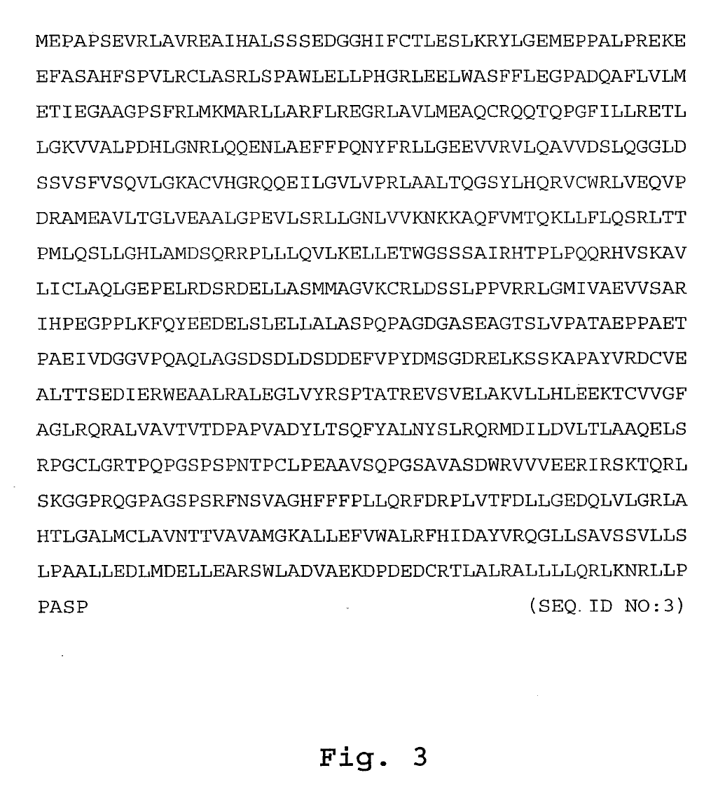

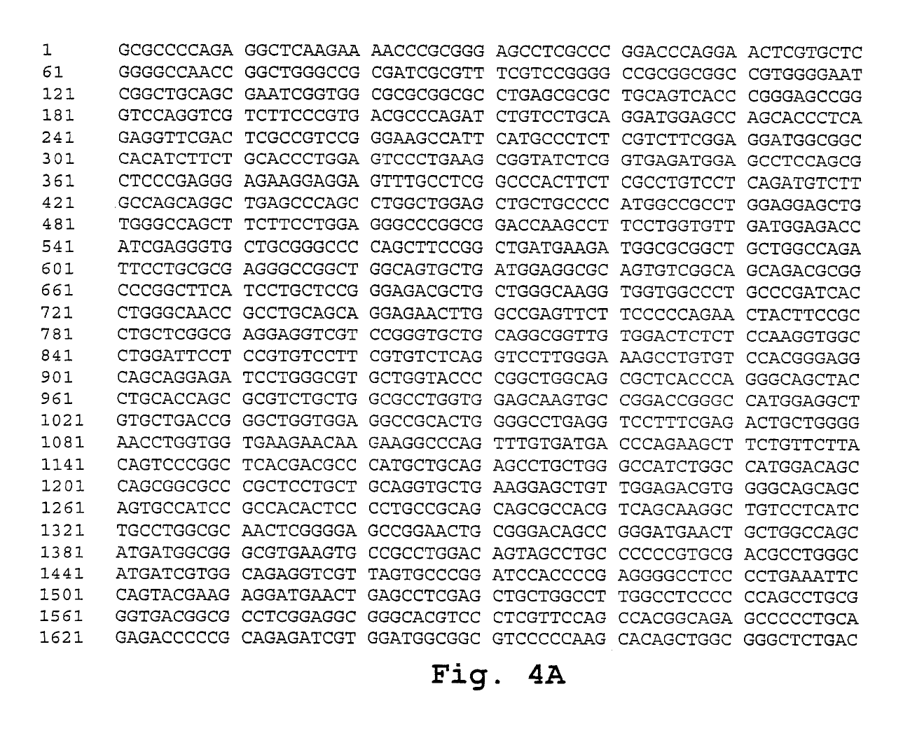

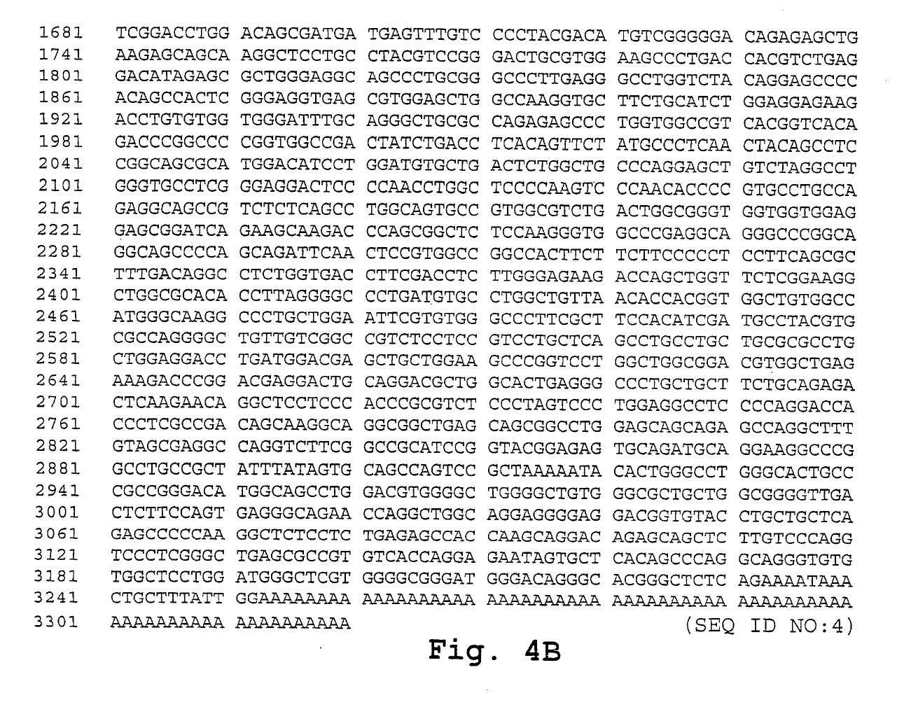

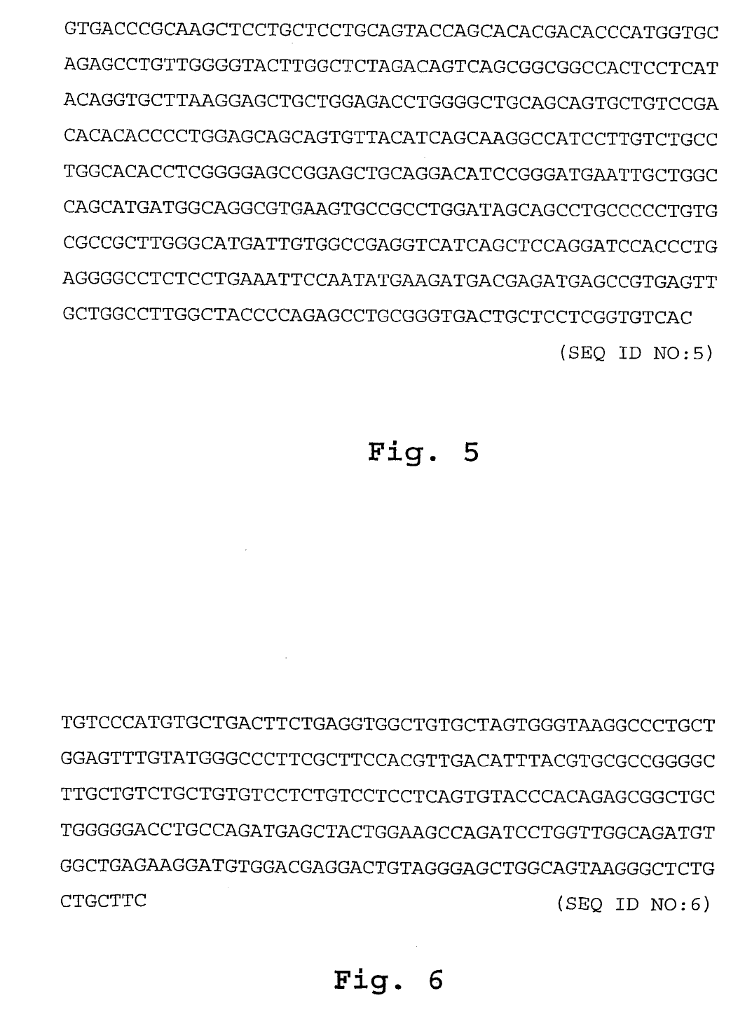

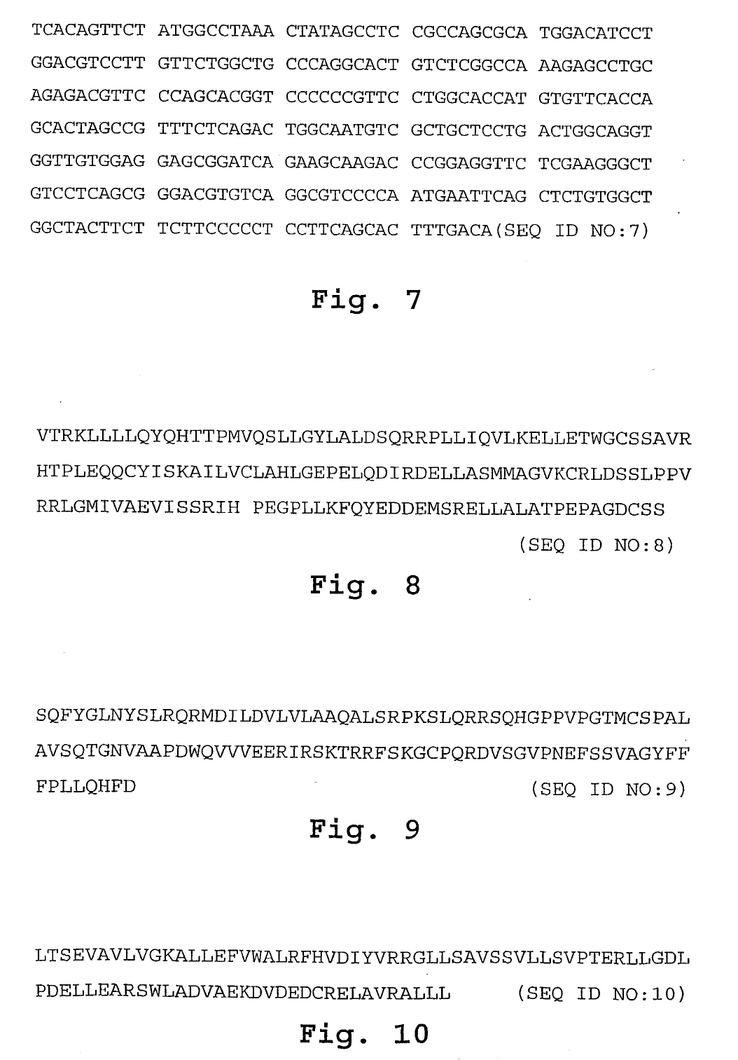

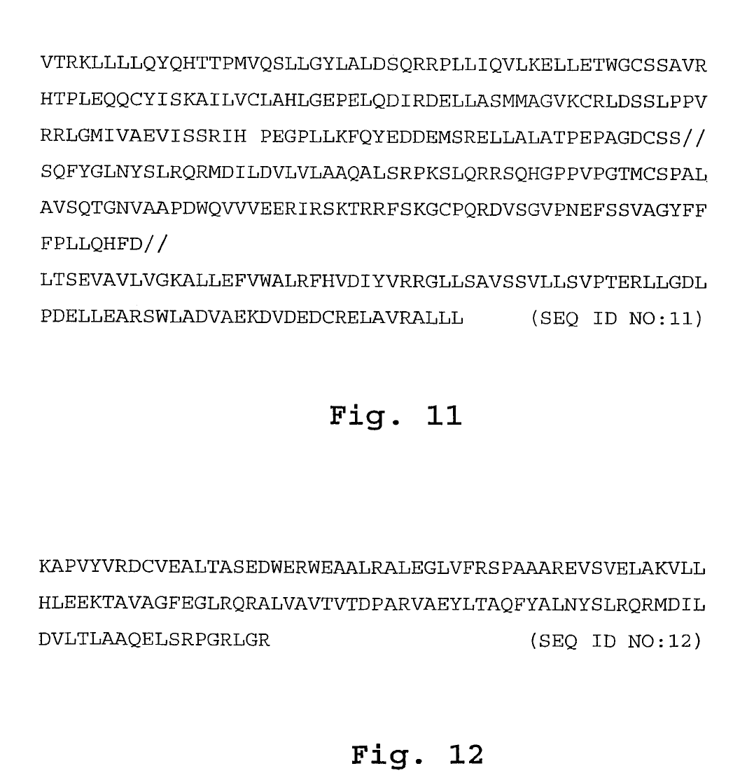

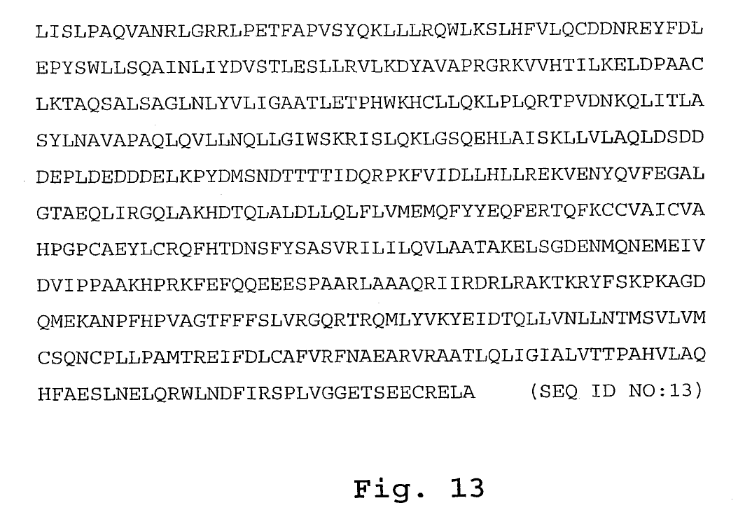

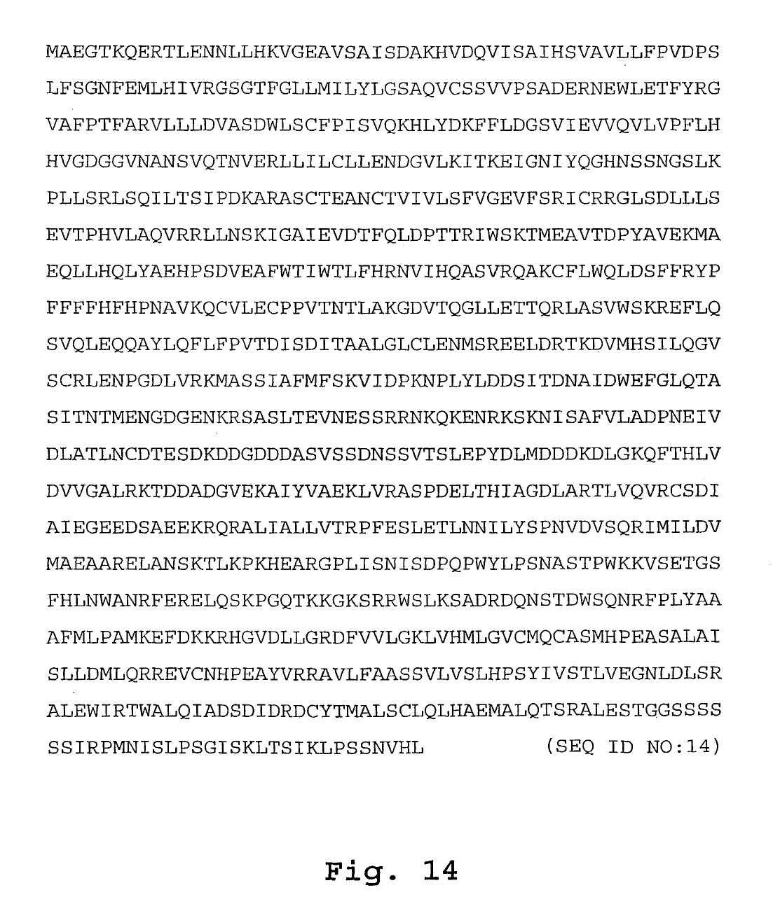

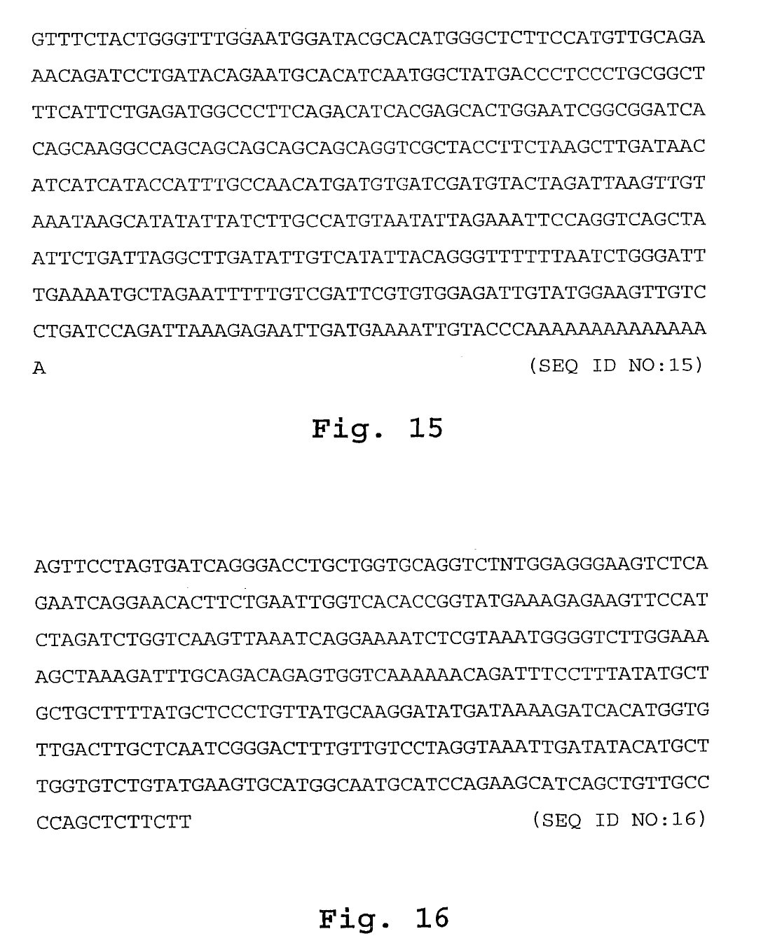

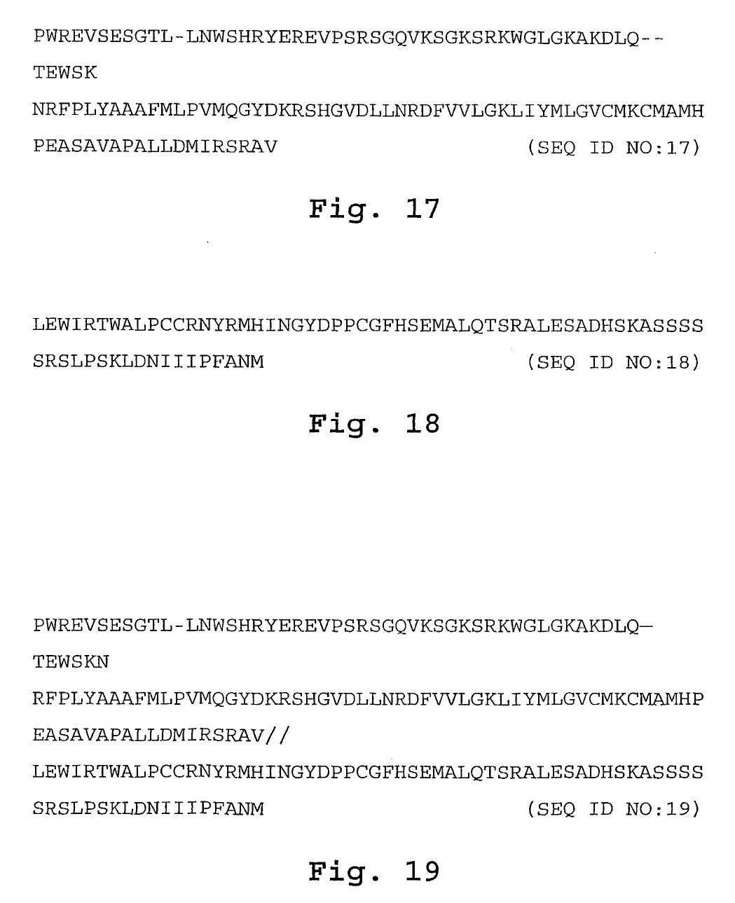

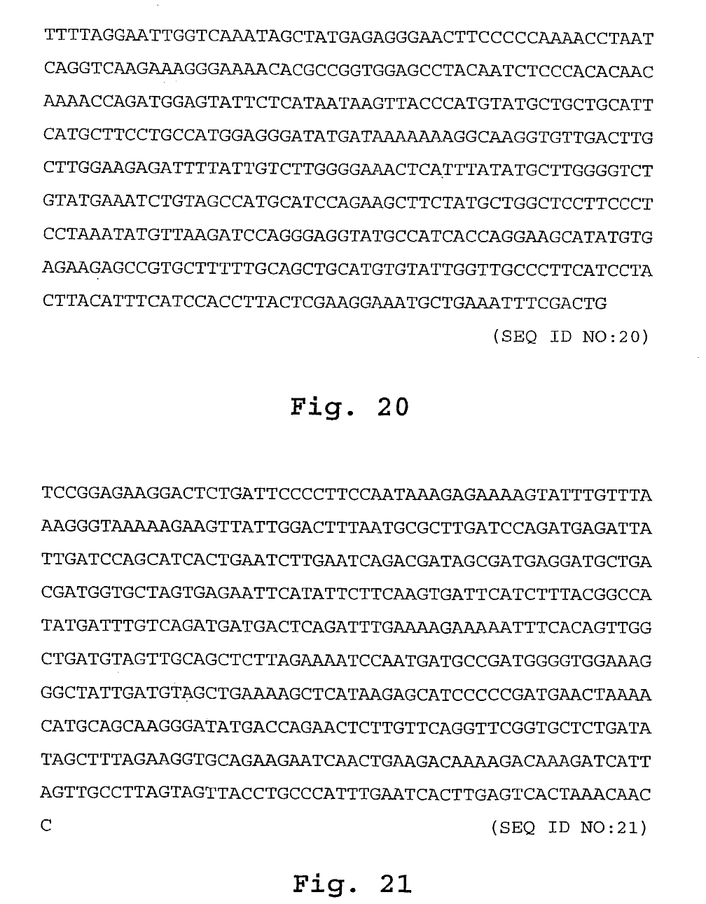

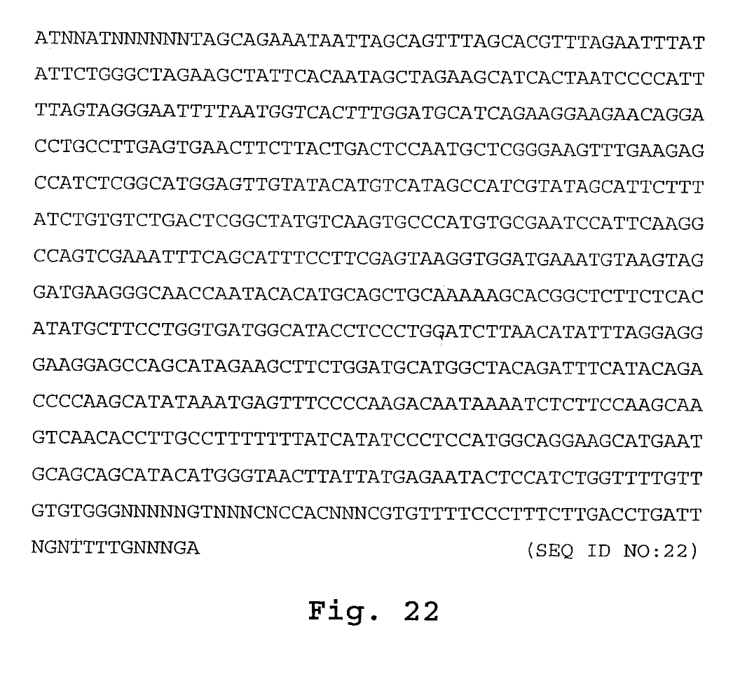

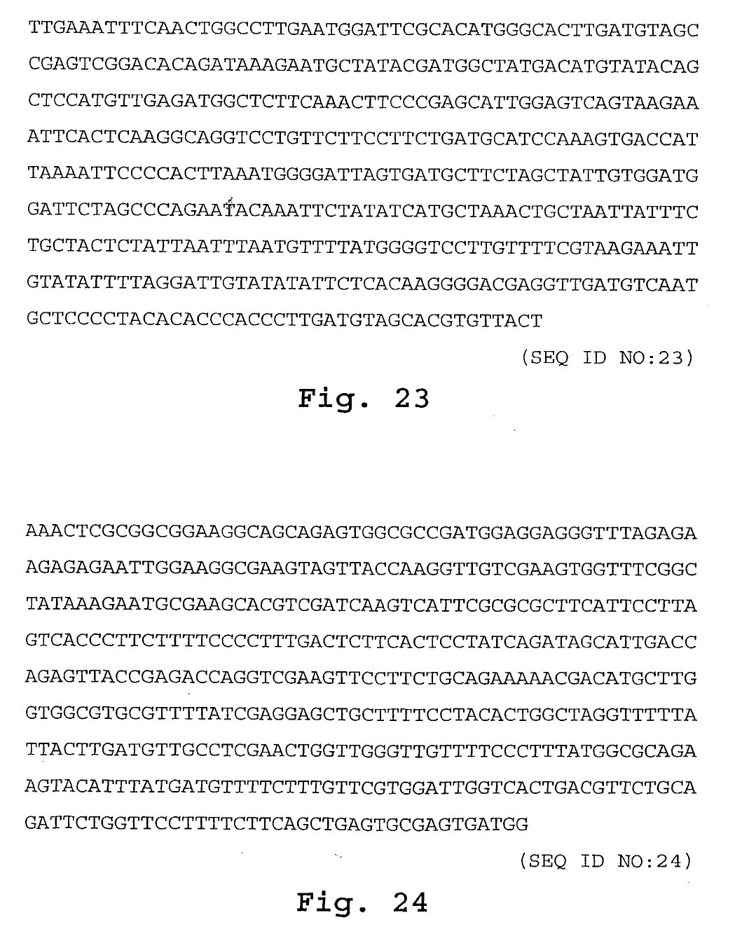

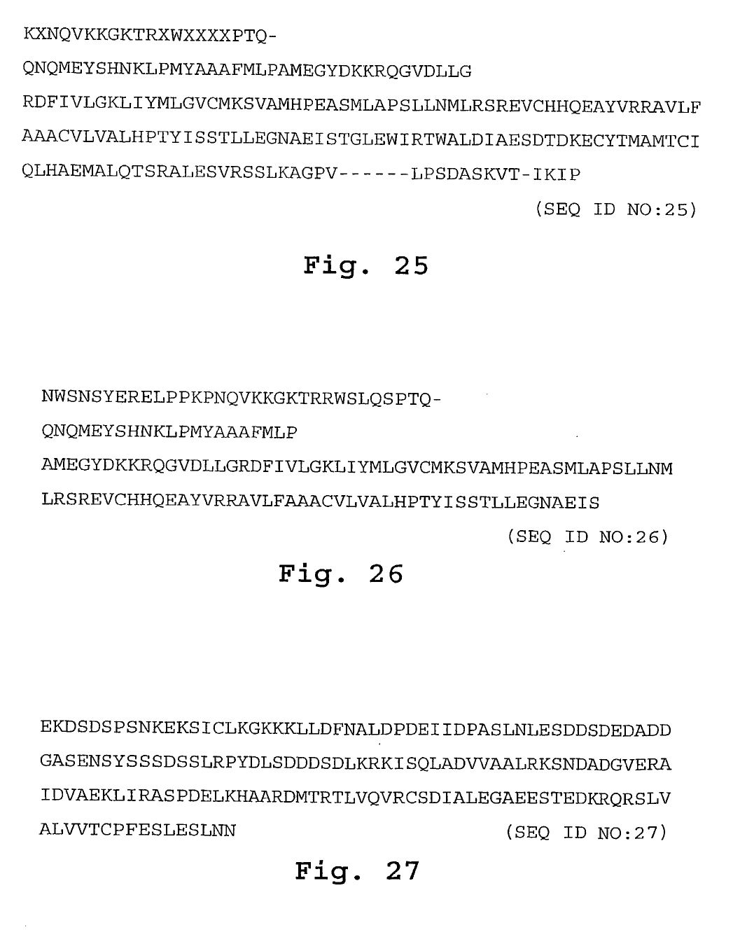

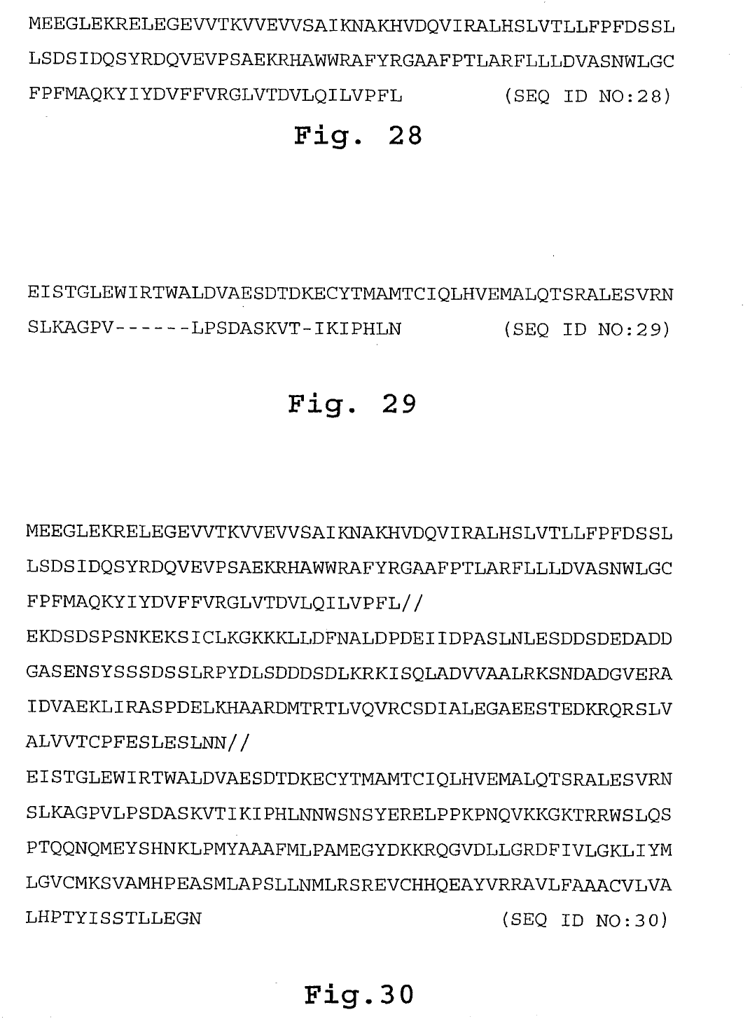

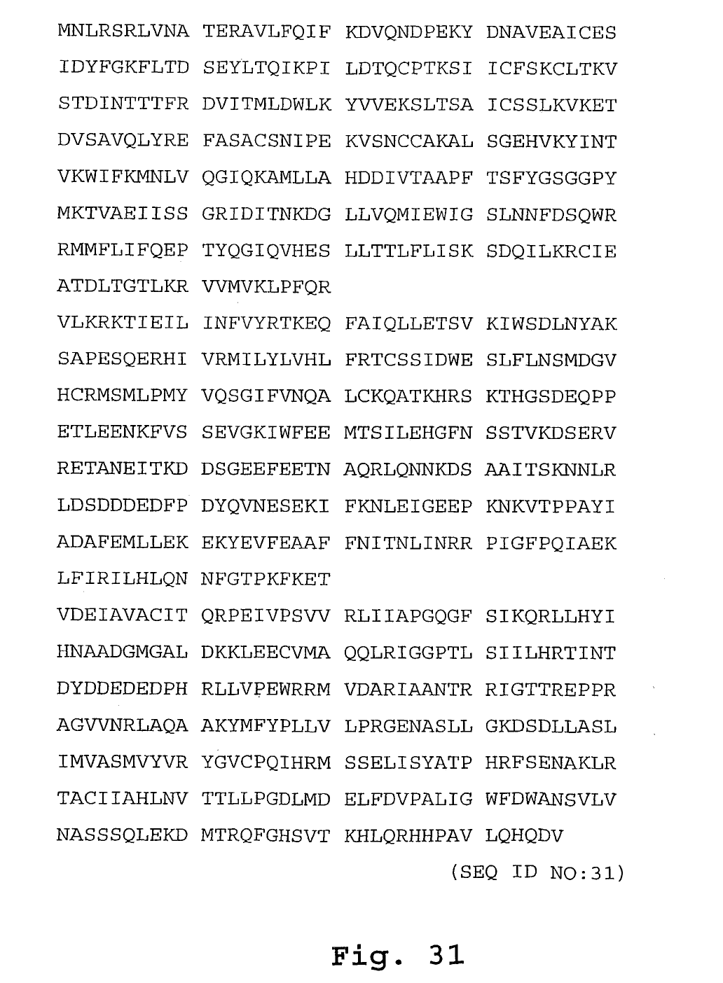

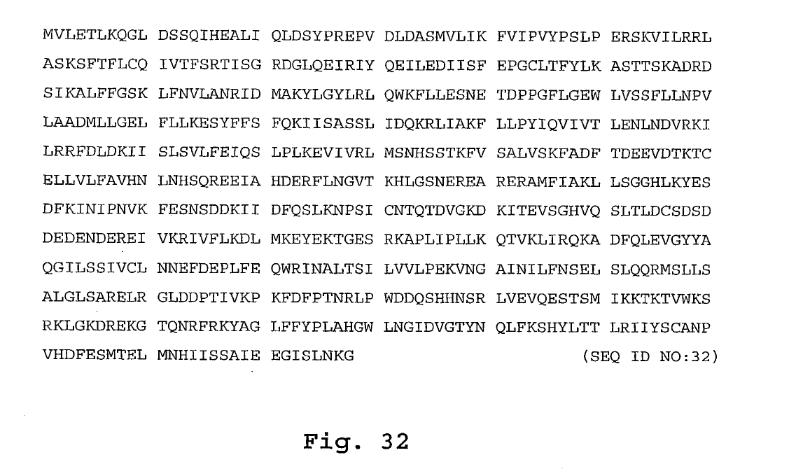

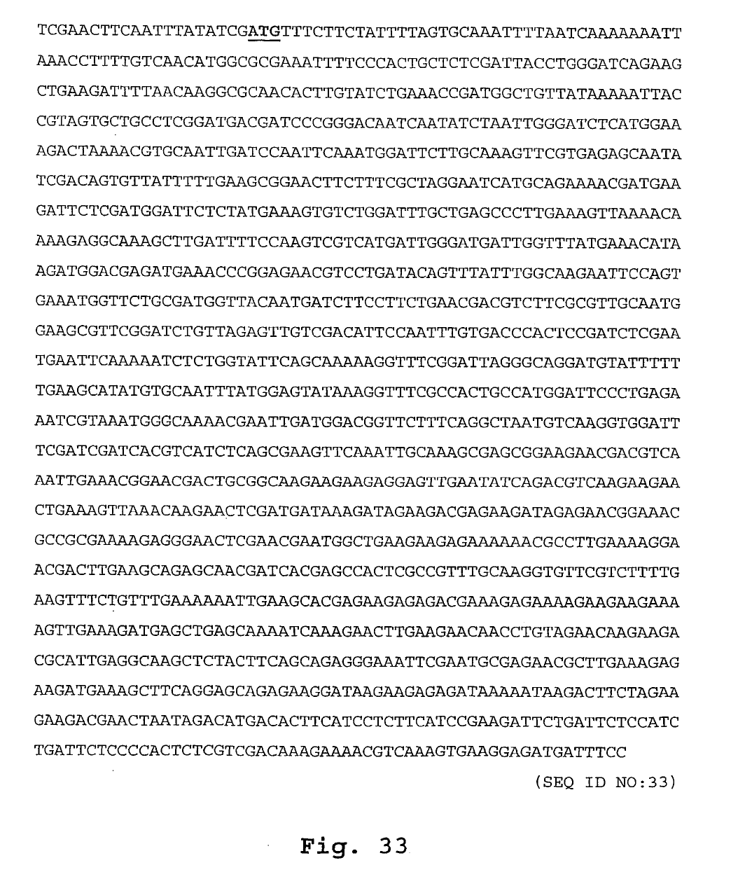

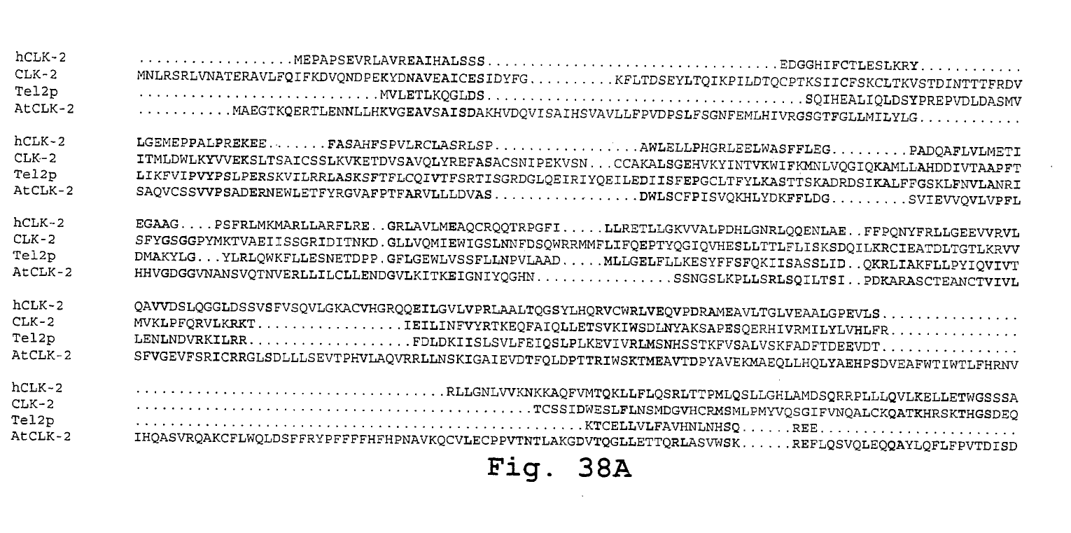

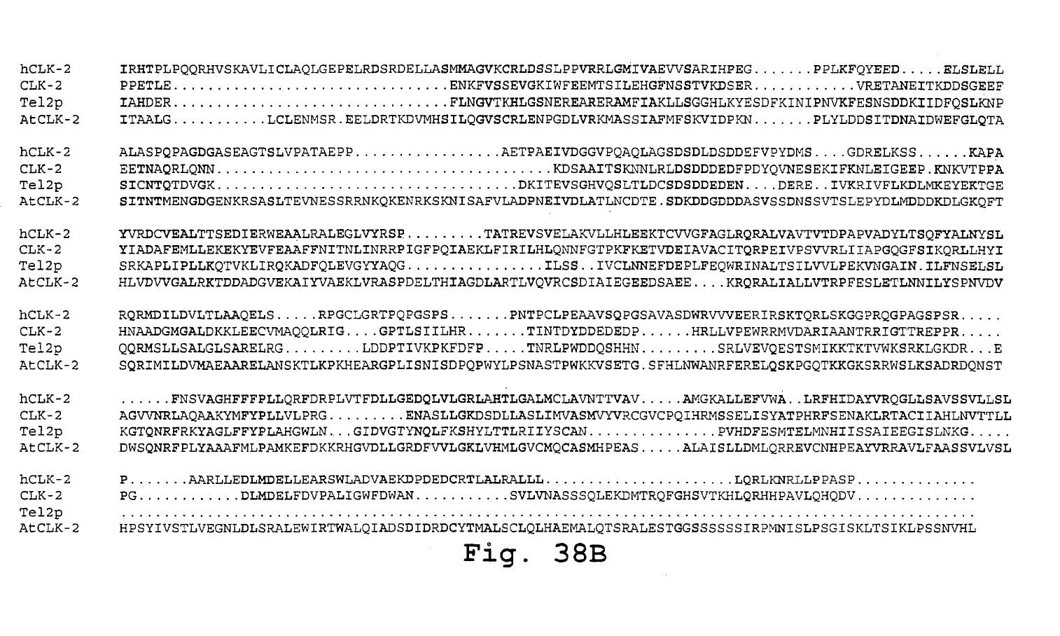

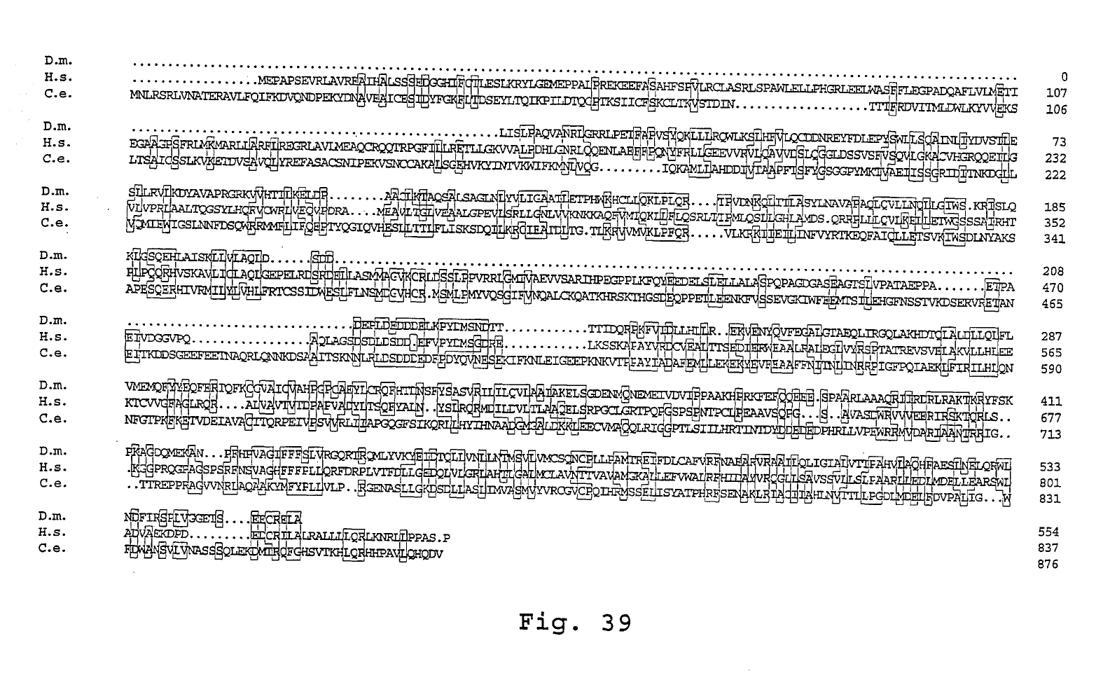

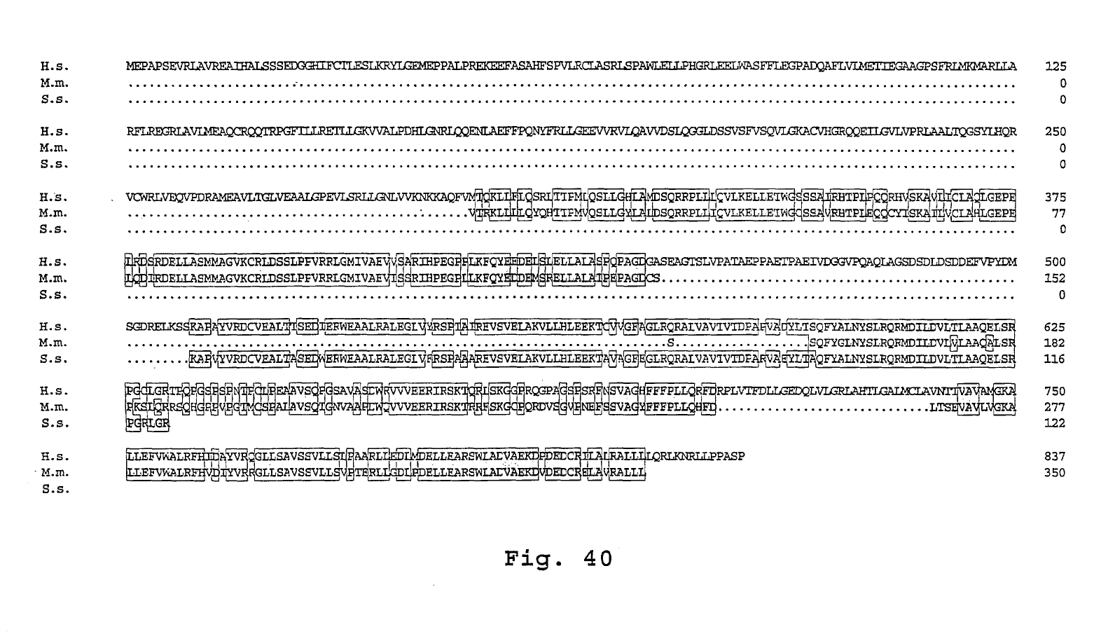

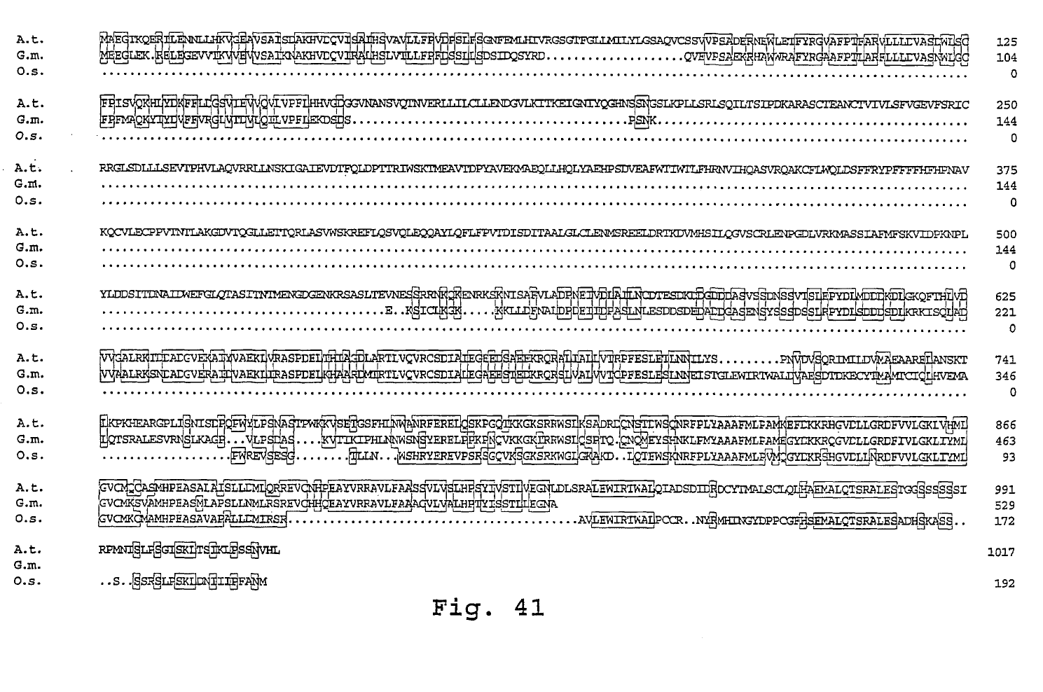

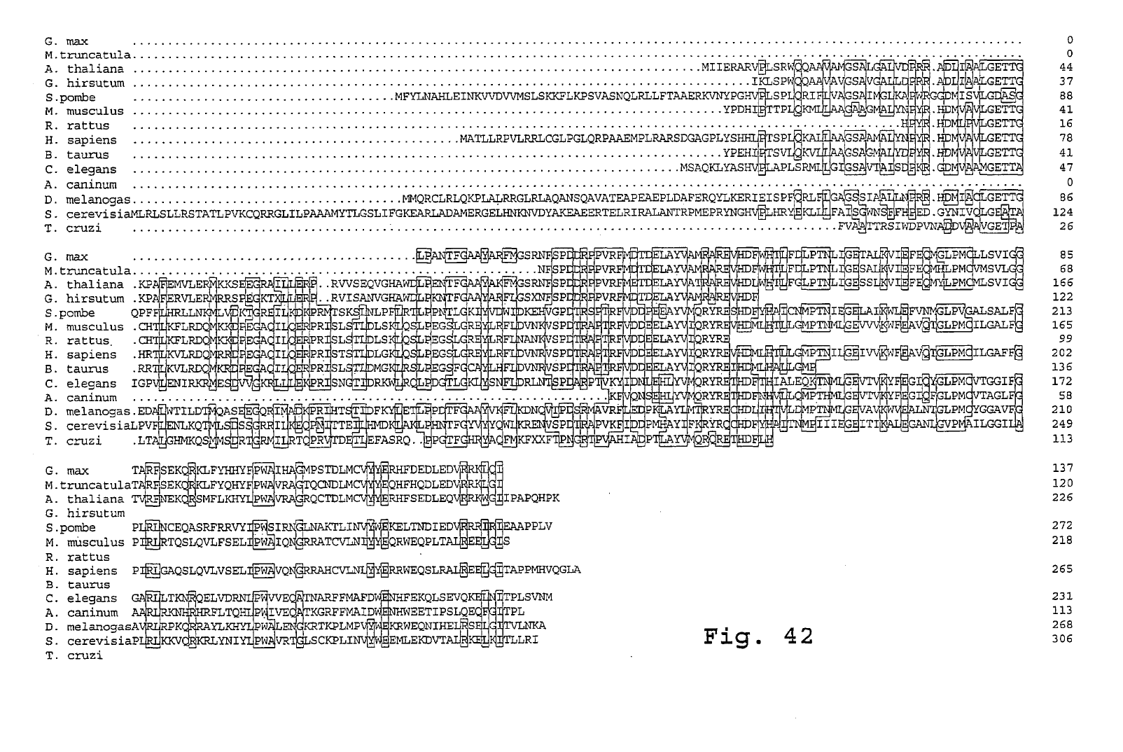

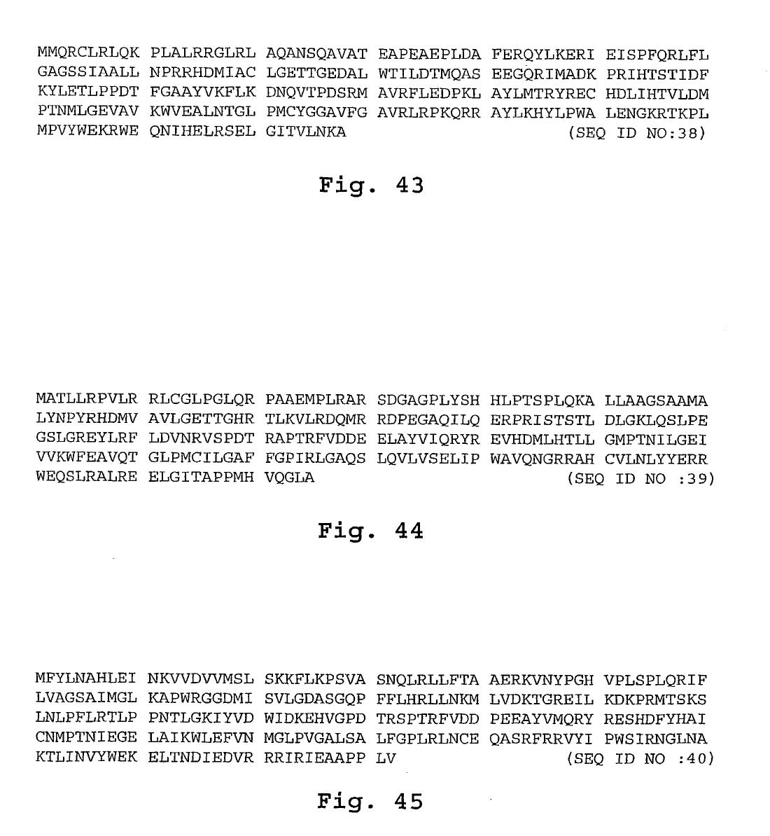

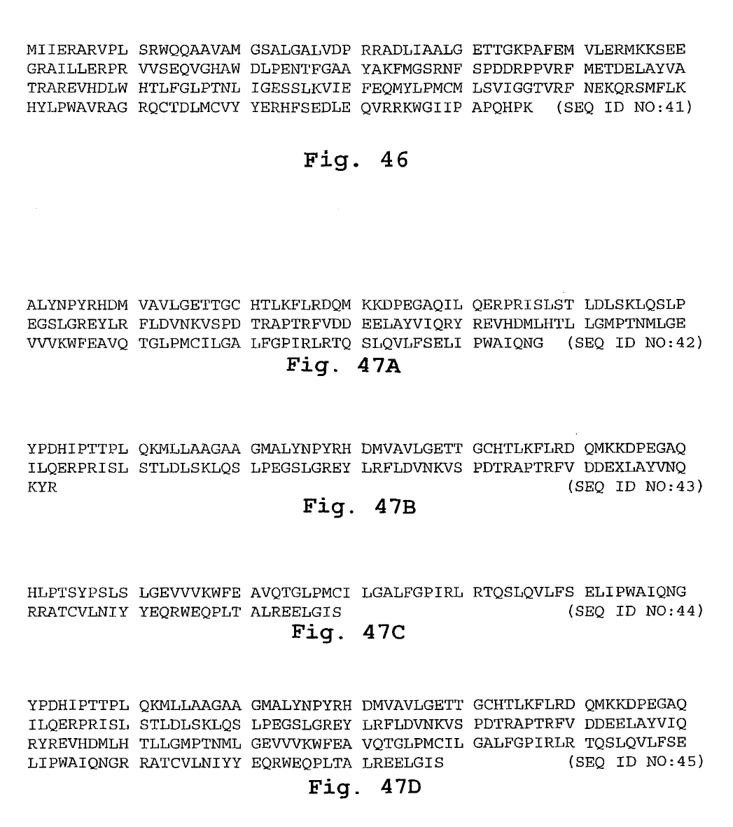

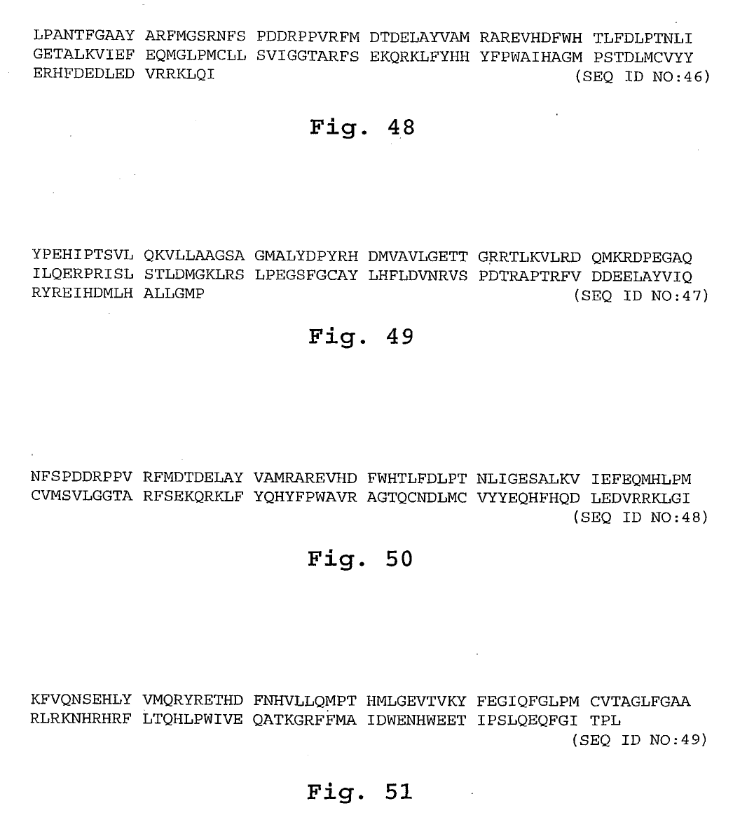

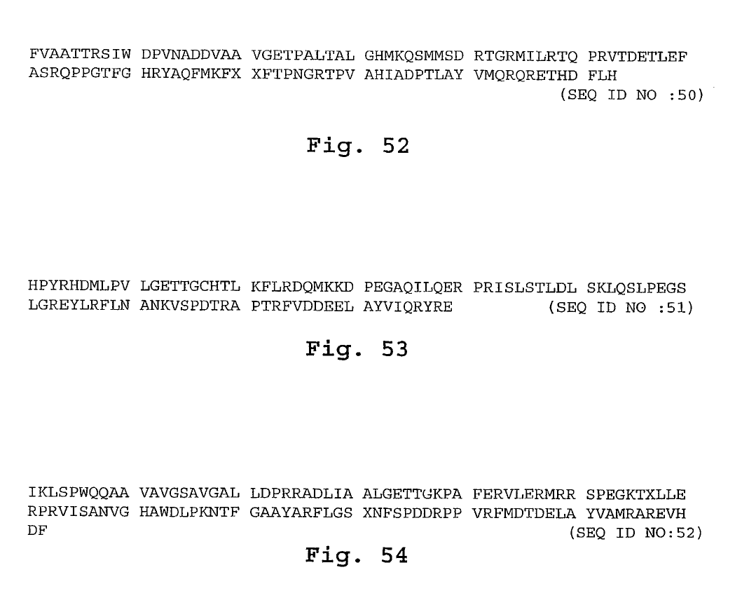

clk-2 encodes a predicted protein of 877 amino acids and the clk-2(qm37) mutation is a cysteine to tyrosine substitution at residue 772 of the predicted protein. We have been able to detect the expressed protein by western blot analysis of protein extracted from both mutant and wild-type worms at different temperatures. CLK-2 is similar to unique predicted proteins in human (FIG. 3), Drosophila (FIG. 13), rice (FIG. 19), soybean (FIGS. 26-30) and to Saccharomyces cerevisiae Tel2p (FIG. 32) and in other species (FIGS. 7-12, 14, 17-19). The structural conservation among these proteins is illustrated by the alignment presented in FIGS. 38, 39, 40 and 41. No homologue of Tel2p had previously been recognized because aligning multiple sequences is necessary to reveal the homology. Tel2p has been shown to bind yeast telomeric DNA in a sequence-specific manner (Kota, R. S. Runge, K. W. Chromosoma 108, 278 (1999); Kota, R. S., Runge, K. W. Nucleic Acids Research 26, 1528 (1998)) and to affect the length of telomeres.

Expression Pattern of clk-2

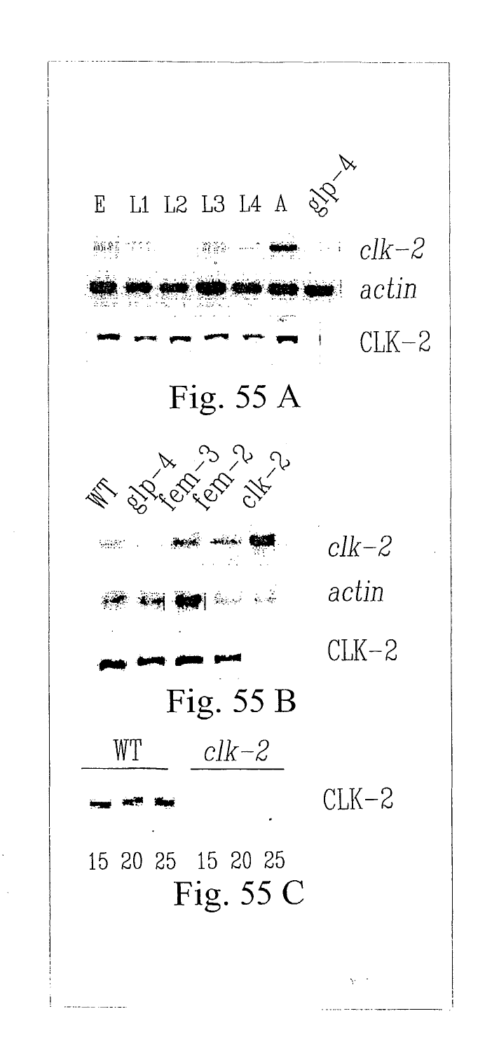

We determined the spatial and temporal expression pattern of the gene clk-2 by analyzing transcript and protein levels (FIG. 55) and by examining transgenic worms carrying reporter fusions. Panel A of FIG. 55 illustrates Northern and Western (37) analyses of clk-2 at all developmental stages. The level of ck-2 mRNA appears uniform throughout pre-adult development (E, embryos; L1-L4, larval stages; A, adult; glp-4, adult glp-4 (bn2ts) mutants at 25° C.). The low level of clk-2 expression in L4 larvae and in glp-4 mutants that lack a germline at 25° C. suggest that most clk-2 RNA in adults is located in gametes. In contrast to the finding with mRNA, the level of CLK-2 protein is similar at all stages including adults (lower panel of A). Panel B of FIG. 55, clk-2 mRNA and protein levels (lower panel) in mutant backgrounds (glp-4 (bn2ts), fem-3 (q20ts), which produces only sperm at 25° C., and fem-2 (b245ts), which produces only oocytes at 25° C.). The mRNA and protein levels of clk-2 expression are similar to the wild type in fem-3 and elevated in fem-2 mutants. glp-4 mutants have wild type protein levels but reduced mRNA levels. clk-2 mRNA appears strongly elevated in clk-2 mutants. Panel C of FIG. 55, CLK-2 protein levels in wild type and clk-2 mutants at three temperatures. clk-2(qm37) is a missense (C772Y) and temperature-sensitive mutation. The level of CLK-2 is greatly reduced in the mutant, but does not change as a function of temperature in either the wild type or the mutant. Worms were raised at 20° C. except when specified otherwise.

We grew populations of worms synchronized at different developmental stages and extracted total or polyA+ selected RNA from them. The highest level of clk-2 mRNA is detected in young adults. We used several mutants to determine the origin of the transcript level in young adults. Since clk-2 mRNA level is highly reduced in glp-4(bn2ts) mutants that do not develop a germline at the non-permissive temperature, most of the RNA present in wild-type young adults is in the germline. Given the low abundance of RNA in L4 larvae which possess an already large germline but only a few male gametes, most of the clk-2 mRNA in wild-type adults is localized to meiotic gametes, in particular to oocytes.

We have analyzed the CLK-2 protein level in different genetic backgrounds and in worms grown at different temperatures. We immunodetected CLK-2 protein on western blots by using two different polyclonal antibodies, MG19 and MG20. We obtained these antibodies by injecting rabbits with a bacterially expressed His₁₀-CLK-2 protein. We found that the content of CLK-2 protein is uniform across developmental stages in wild type and in clk-2 animals. Furthermore, the concentration of CLK-2 is not different from the wild type in, glp-4 mutants which have no germline, nor in fem-3 and fem-2 mutants that contain only sperm and only oocytes, respectively. Taken together these results indicate that gametes specifically accumulate high levels of clk-2 mRNA, presumably as a store to be used by the embryo. Finally, we observed that in qm37 mutants, while the level of clk-2 mRNA appears slightly elevated, the level of CLK-2 protein is greatly reduced.

We constructed three reporter constructs of the clk-2 gene that comprised different upstream promoter regions and/or the coding region of the clk-2 gene fused to the green fluorescent protein. Two of the constructs are transcriptional fusions, one containing bases 36932 to 37319 and the other containing bases 36932 to 40010 of cosmid C07H6 [Accession: AC006605]. A third reporter construct (pMQ251) is a translational fusion that contains bases 30501 to 37319, except bases 35078 to 36545 which are part of the gene cex-7. We microinjected these reporter genes into wild type and clk-2(qm37) mutant worms, and analyzed numerous worms from several transgenic lines carrying these reporters. We observed that the clk-2 promoter region directs expression in all somatic tissues, including hypodermis, muscles, neurons, excretory system, gut, pharynx, somatic gonad, vulva, and presumably all cells. No expression was visible in the germline, despite the use of both standard and complex array mixes. This is commonly the case for transgenes in C. elegans and does not indicate an absence of expression in the germline tissue. A full length fusion protein between CLK-2 and GFP (encoded by the construct pMQ251) that complements the mutant phenotype for development, behavior and viability at 25° C., is localized exclusively into the cytoplasm, which is consistent with the absence of an obvious nuclear localization signal in the predicted protein. The pattern observed is not a consequence of overexpression as very small transgene concentrations have been used in complex arrays (Kelly et al., Genetics 146:227-238, 1997). However, although the nucleus appears dark in the fluorescent images, it still may contains very small amounts of the fusion protein. This analysis of expression indicates that CLK-2 protein is indeed produced in the nematode, as shown by western analysis on total C. elegans extracts using anti-CLK-2 antibodies.

Yeast Tel2p has been found to bind telomeric repeats in vitro, and thus is expected to be nuclear in vivo. However, it was found that CLK-2::GFP is excluded from the nucleus. Subtelomeric silencing and telomere length regulation can also be affected by events in the cytosol. For example, Hst2p, a cytosolic NAD+-dependent deacetylase homologous to Sir2p, can modulate nucleolar and telomeric silencing in yeast Perrod et al., EMBO J., 20(Nos 1 & 2), 197-209, 2001), and the nonsense-mediated mRNA decay pathway appears to affect both telomeric silencing and telomere length regulation (Lew et al., Molecular and Cellular Biology, 18(10):6121-6130, 1998). Other proteins that affect telomere length, like tankyrase Smith, S. and De Lange, Titia, J. of cell Science, 112:3649-3656, 1999), are mostly extranuclear Chi, N.-W., and Lodish, H. F., J. of Biological Chemistry, 275(49):38437-38444, 2000), with only a very small amount of protein localized to the telomeres Smith et al., Science 282:1484-1487, 1998).

The Role of clk-2

Telomere function has been found to affect replicative life span in yeast and in vertebrate cells. It also has also been shown to affect the immortality of the germline in C. elegans. However, an involvement of telomere function in determining the life span of muiticellular organisms has not been established prior to this work. Here we have shown that the maternal-effect clk-2 gene of C. elegans regulates telomere length, and prolongs life span by a mechanism that is distinct from the regulation of dauer formation but resembles caloric restriction, and encodes a protein that is similar to the yeast telomere binding protein Tel2p.

The timing of the lethal action of clk-2 (qm37) indicates a function for clk-2 during the events that immediately follow fertilization, including oocyte meiosis, pronuclei formation and karyogamy, and this would be consistent with the known importance of telomeres in meiosis. However, our examination of the morphology of chromosomes in oocytes and early embryos did not reveal any abnormalities. Similarly, although telomere function appears linked to double strand break repair and chromosome stability, including in worms, clk-2 mutants appear only moderately sensitive to ionizing radiation and do not display signs of chromosome instability. In fact, we examined the response of clk-2 (qm37) mutants to gamma-radiation and found that among the progeny of irradiated animals, the proportion of dead eggs and larvae was about 10 times higher than among the progeny of irradiated wild-type animals. There is also no report of a function of Tel2p in the response to ionizing radiation in yeast.

The null phenotype of tel2 is lethal but a hypomorphic mutation of tel2 results in short telomeres and slow growth (Runge, K. W. and Zakian, V. A. Molecular & Cellular Biology 16, 3094 (1996). Tel2p has been shown to be involved in telomere position effect (TPE) and thus contributes to silencing of sub-telomeric regions (Runge, K. W. and Zakian, V. A. Molecular & Cellular Biology 16, 3094 (1996), one of the best studied examples of epigenesis. Mutations in other genes, such as tell, that also result in telomere shortening do not result in abnormal TPE, indicating that the TPE defect in tel2 mutants is not a simple consequence of short telomeres. Furthermore, the rapid death and abnormal cellular morphology of cells fully lacking Tel2p suggests that Tel2p, like Rap1p and the Sir proteins, also functions at non-telomeric sites (Zakian, V. A. Ann. Rev. Genet. 30, 141 (1996)). In light of this, the absolute requirement for maternal clk-2 in embryogenesis suggests a function for CLK-2 in silencing genes that-are needed during some part of the worm's life cycle but that are deleterious when expressed during early development. The study of the mes genes which are required for the specification of the germline in C. elegans and can confer maternal-effect sterile phenotype has shown that mechanisms of silencing are part of the normal development of worms. Indeed, some of the mes genes have been found to encode proteins that resemble Polycomb group proteins and appear generally to be involved in the regulation of chromatin structure.

Mutations in clk-1 and clk-2(qm37) at the permissive temperature confer a similar CLK phenotype and in particular an increase of life span of similar magnitude (Lakowski, B. and Hekimi, S. Science 272, 1010 (1996) and show similar pattern of interactions with other aging genes (Lakowski, B. Hekimi, S. Proc. Nat. Acad. Sci. US 95, 13091 (1998)). CLK-1 is a mitochondrial protein of unknown function (Felkai, S. et al, EMBO Journal 18, 1783 (1999).). In an attempt to explain many puzzling features of the clk-1 phenotype, including the maternal effect, we have suggested that the action of CLK-1 is to indirectly, but specifically, regulate nuclear gene expression (Branicky R, C. Benard, S. Hekimi, Bioessays 22:48, 2000). One possibility might be that CLK-2 might be one of the molecules that implements changes in gene expression in response to alteration of CLK-1 activity. clk-1 clk-2 double mutants have a phenotype that is more severe than either of the single mutants (Lakowski, B. and Hekimi, S. Science 272, 1010 (1996). However, the phenotype of a double mutants containing the null allele clk-1 (qm3O) is not more severe than a double mutant containing the much weaker allele clk-1 (e2519), in contrast to the situation with clk-3, for which double mutants with clk-l(qm30) are much more severe than with clk-1 (e2519) (Lakowski, B. and Hekimi, S. Science 272, 1010 (1996). These observations indicate that at least part of the activity of clk-1 requires clk-2. Furthermore, clk-1 clk-2 double mutant embryos resemble clk-1 mutant in that the interphases of the embyronic cell cycles are slowed down, but mitoses appear unaltered. This indicates that clk-2 as well as clk-1 is involved in determining the rate of cellular multiplication, and thus affects mechanisms which are known to lead to cancer when deregulated.

Telomere function has also been implicated in the replicative life span of yeast, where Sir proteins mediate silencing at the telomeres and the HM loci. When displaced from the telomeres by mutation or by shortage of telomeric DNA, part of the Sir complex can move to the nucleolus where its action appears to prolong replicative life span. These and other studies indicate that telomeres are a reserve compartment for silencing factors and participate in regulating silencing in other parts of the genome. It has been suggested that the effect on cellular senescence of expressing telomerase in cultured human cells might be mediated by an effect on silencing rather than by preventing chromosome erosion. Therefore, clk-2 must be involved in determining cellular senescence, including in vertebrates, and affect in this manner aging and diseases linked to cellular senescence such as cancer.

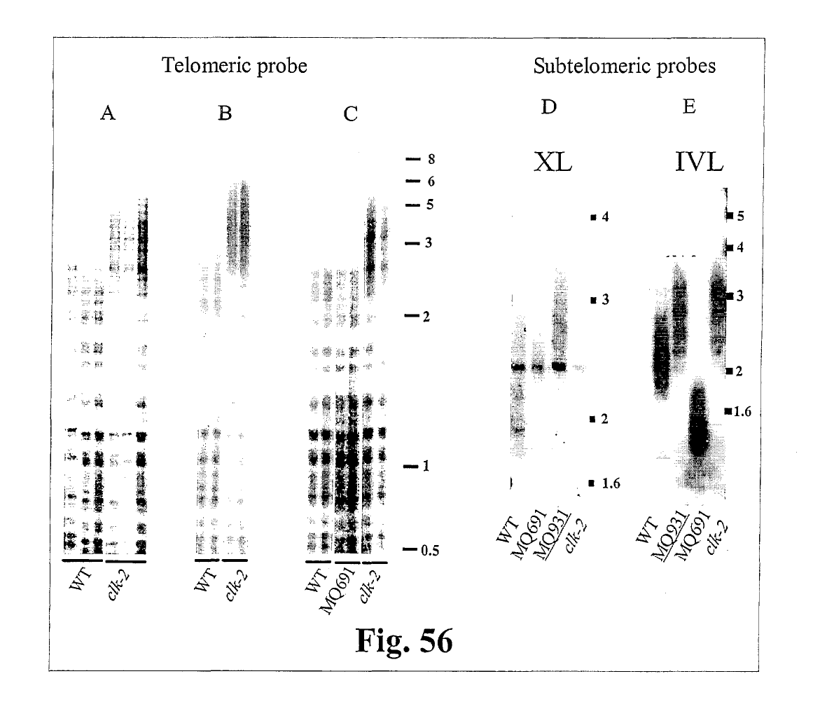

As mentioned earlier, CLK-2 is similar to predicted proteins in vertebrates and plants as well as to Saccharomyces cerevisiae Tel2p. Tel2p has been shown to bind yeast telomeric DNA in a sequence-specific manner, and to affect the length of telomeres. We found that clk-2 also affected the length of telomeres in worms (FIG. 56). In worms, genomic DNA hybridization to telomeric probes after restriction digestion with HinfI reveals the end fragments of the chromosomes carrying the telomeres, which appear as smears, as well as fragments carrying tracts of telomeric repeats that are internal to the chromosome, which appear as discrete bands. The regions where the telomeric smears are the most intense are indicated by stippled lines. Two lanes are shown for each genotype and each temperature.

The length of telomeres in wild-type and clk-2 mutants was examined by Southern blotting at three temperatures, including the lethal temperature. For 18 and 20° C., worms were grown for numerous generations at each temperature before DNA extraction. Since clk-2(qm37) is lethal at 25° C., mixed stage worms from 20° C. were transferred to and grown at 25° C. for 3-4 days. Genomic DNA was prepared, HinfI digested and separated on a 0.6% agarose gel at 1.2Vcm⁻¹. Southern blots were hybridized with gamma ³²P DATP end-labelled TTAGGCTTAGGCTTAGGCTTAGGCTTAGGCTTAGGCTTAGGCTTAGG oligo-nucleotide. Use of a second type of probe, made by direct incorporation of alpha ³²P DATP during PCR amplification of telomeric repeats from the plasmid cTel55X with primers T7 and SHP1617 (GAATAATGAGAATTTTCAGGC), gave identical results. The extrachromosomal array in MQ691 clk-2(qm37); qmEx159 contains a clone with the entire coding sequence of clk-2 as well as the promoter of the operon but excluding cux-7 (bases 37319 to 31528 of cosmid C07H6, except bases 36544 to 35077) and rescues clk-2 mutant phenotypes. In clk-2 mutants, telomeres are two to three times longer than in the wild type on average (FIG. 56). However, the chromosomes are of wild-type length in strain MQ691, which carries an extrachromsomal array expressing wild-type CLK-2 in a clk-2(qm37) chromosomal background (FIG. 56) indicating that the alteration of telomere length clk-2 (qm37) mutants is indeed due to abnormal function of clk-2 in these mutants.

The length of terminal telomeric fragments in the animals of the strain MQ691, which carries an extrachromosomal array (qmEx159) containing functional wild-type CLK-2 that rescues development and behavior at 25° C. in a clk-2(qm37) chromosomal background, was further analyzed. A similar clone containing the qm37 mutation fails to rescue the Clk-2 phenotypes. In MQ691 animals, the length of terminal telomeric fragments appear very similar to the wild-type, and even shorter, indicating that the lengthened telomere phenotype of qm37 mutants is rescued by the expression of clk-2(+). The telomere length of non-transgenic animals of the strain MQ931, derived from MQ691, which have lost the extrachromosomal array and thus again lack clk-2(+) has been further examined. The terminal telomeric repeats in this strain are long again. Thus, the lengthened telomere phenotype of clk-2(qm37) can be rescued by clk-2(+) and reverses back to mutant length after the loss of the transgene.

In C. elegans, tracks of numerous TTAGGC telomeric repeats are present at the ends of the 6 chromosomes (Wicky C., et al., Proc. Natl. Acad. Sci. USA, 93:8983-8988, 1996). In addition, numerous interstitial blocks of perfect and degenerate telomeric repeats are located more internally to the chromosomes (C. elegans II. Edited by Riddel D et al. Published by Plainview, N.Y.: Cold Spring Harbor Laboratory Press (1997), pp 56-59, Chapter 3). Analysis of genomic DNA after restriction digestion with a frequent cutter that does not cleave within the telomeric repeats (HinfI), electrophoresis, and hybridization to telomeric probes, reveals the telomere-carrying end fragments of the chromosomes (Wicky C., et al., Proc. Natl. Acad. Sci. USA, 93:8983-8988, 1996). Telomeres, and thus the restriction fragments containing them, are heterogeneous in size and appear as smears. On the other hand, restriction fragments carrying tracts of internal telomeric repeats are of fixed size and appear as discrete bands in the 0.5-3 kb range (Ahmed S, Hodgkin J. Nature, 403(6766):159-64, 2000; and Wicky C., et al., Proc. Natl. Acad. Sci. USA, 93:8983-8988, 1996). The quality of visualization of the length of telomeres in C. elegans with a hybridization probe that detects telomeric repeats is marred by the numerous internal repeats that also hybridize to the probe. In particular, they can mask the detection of the telomeres of chromosomes that have small HinfI terminal telomeric fragments. To further describe the telomere phenotype of clk-2(qm37) mutants, the length of individual telomeres has been characterized. The subtelomeric regions just adjacent to the terminal telomeric repeats share no sequence homology among the chromosomes (Wicky C., et al., Proc. Natl. Acad. Sci. USA, 93:8983-8988, 1996). Taking advantage of this sequence diversity, probes specific to particular telomeres were designed. The size of a given HinfI terminal fragment is related to the fixed distance between the most exterior HinfI site of the chromosome and the beginning of the telomeric repeats, and by the variable number of terminal telomeric repeats. Upon genomic DNA digestion with HinfI and Southern blotting with a probe specific to a particular telomere, the terminal fragments, which are heterogeneous in size, again appear as a smear. Detailed results obtained for two individual telomeres are illustrated in FIG. 56.

The length of the terminal fragment of the left telomere of chromosome X is ˜1 kb longer in qm37 than in the wild type, ranging from 2.4 to 4.2 kb and from 1.7 to 2.8 kb, respectively. This telomere is of wild-type length in MQ691, which carries the rescuing transgene, and lengthens again to the clk-2(qm37) values in the non-rescued MQ931 strain. The length of another terminal fragment (left telomere of chromome IV) is also ˜1 kb longer in qm37 than in the wild type, ranging from 2.2 to 3.9 kb and from 1.8 to 2.8 kb respectively. This telomere becomes shorter than the wild type in MQ691, ranging from 1.3 to 2 kb only. This telomere acquires the mutant length again after loss of the transgene in MQ931. Thus, the overexpression of clk-2 can shorten the tracks of telomeric repeats, but not at each telomere.

Identity of the Gene cog-4 and the cog-4 (qm143) Mutants

The gene COQ7/CAT5 of the yeast S. cerevisiae is the homologous gene to clk-1 (Ewbank, J. J. et al, Science 275, 980 (1997); PCT/CA97/00768). While Coq7p does not structurally resemble an enzyme, it is required for ubiquinone biosynthesis in yeast. A second gene, COQ4 (Marbois, B. N. and Clark, C. J Biol Chem, 271, 2995 (1996) (Accession: NP_{—}010490), that is also required for ubiquinone biosynthesis in yeast, does not code for an enzyme, and like COQ7, has no homologue in bacteria. We have generated a deletion mutant worm to describe the role of the gene coq-4 and its functional relationships with the clk genes we have identified and described, including clk-1, clk-2, and gro-1.

The gene coq-4 in C. elegans largely corresponds to the predicted gene T03F1.2 of the cosmid T03F1 (Accession U88169). It is localized on LGI, between unc-73 and unc-11. coq-4 is less than 100 kb away from the characterized gene, unc-73, and less than 40 kb away from the other characterized gene, unc-11. coq-4 is 843 bp long and has four exons. We experimentally established the structure of the gene coq-4 by sequencing a cDNA clone, yk140a2. A second gene, T03F1.3, which is highly similar to phosphoglycerate kinase (PGK), is 264 bp upstream of coq-4 and, as we have shown, forms an operon with coq-4 and is thus transcriptionally co-expressed. We showed that Coq-4 is in the same operon as T03F1.3 by RT-PCR, that coq-4 is SL2 trans-spliced and that T03F1.3 is SL1 trans-spliced.

We have generated a coq-4-(qm143) deletion mutant by carrying out PCR-based mutant screen following a large scale EMS mutagenesis wild-type worm. coq-4 (qm143) has a 1469 bp deletion, which starts from 44 bp downstream of T03F1-3, and ends 406 bp downstream of coq-4. The predicted gene downstream is 1521 bp away from coq-4 and 1115 bp away from the deletion. Therefore, coq-4 (qm143) is a null mutant and it does not affect the coding sequence of any gene other than coq-4.

The Phenotype of cog-4 Mutants

cog-4(qm143) is a non-strict maternal-effect lethal mutation. Most of the progeny, from a homozygous coq-4 hermaphrodite, dies during embryogenesis. Very few eggs hatch, and those which do hatch fail to complete development and die as young larvae. We have also shown that maternal cog-4 product is sufficient for homozygous coq-4 to develop normally until adulthood. However, homozygous coq-4 adult worms from a heterozygous hermaphrodite (coq-4/+) are paralytic and are defective in egg-laying. Moreover, coq-4 homozygous mutants can be mated by N2 males and produce progeny, which grow normally. Taken together, these results indicate that either maternal or zygotic coq-4 product is sufficient for coq-4 mutant to go through embryonic and post-embryonic development. coq-4 deletion (qm143) is kept as a balanced strain, coq-4(qm143)/unc-73(e936). We demonstrated that the phenotypes of the coq-4 mutants, in particular the sterility, can be rescued by an extrachromosomal wild-type copy of coq-4 DNA fragment.

Expression Pattern of cog-4

The spatial expression pattern of coq-4 was determined by using translational reporter fusion to the green fluorescence protein, containing 2.2 kb of upstream promoter region. These constructs were injected into both N2 and heterozygous coq-4 (coq-4/unc-73), and animals of several transgenic lines were examined. We found that a functional coq-4::gfp is expressed in the hypodermis, muscles, the gut, the excretory canal and embryos. In addition, we detected that the reporter fusion localizes to the mitochondria, in particular, in muscle cells.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A clk-2 gene which has a function at the level of cellular physiology involved in developmental rate, telomere length and longevity, wherein clk-2 mutations cause a longer life, an altered cellular metabolism and/or an altered telomere length relative to the wild type, wherein clk-2 overexpression leads to telomere shortening, and wherein clk-2 gene has the identifying characteristics of nucleotide sequence set forth in SEQ ID NO:1.

2. Use of a clk-2 gene to alter a function at the level of cellular physiology involved in the regulation of developmental rates, telomere length and longevity, wherein clk-2 mutations cause a longer life, altered cellular metabolism and physiological rates and/or an altered telomere length relative to the wild type, wherein clk-2 overexpression leads to telomere shortening, and wherein clk-2 gene has the identifying characteristics of nucleotide sequences set forth in SEQ ID NO:1, SEQ ID NO:4, SEQ ID NO:5, SEQ ID NO:6, SEQ ID NO:7, SEQ ID NO:15, SEQ ID NO:16, SEQ ID NO:20, SEQ ID NO:21, SEQ ID NO:22, SEQ ID NO:23, or SEQ ID NO:24, or wherein said gene codes for a protein sequence as set forth in SEQ ID NO:2, SEQ ID NO:3, SEQ ID NO:8, SEQ ID NO:9, SEQ ID NO:10, SEQ ID NO:11, SEQ ID NO:12, SEQ ID NO:13, SEQ ID NO:14, SEQ ID NO:17, SEQ ID NO:18, SEQ ID NO:19, SEQ ID NO:25, SEQ ID NO:26, SEQ ID NO:27, SEQ ID NO:28, SEQ ID NO:29, SEQ ID NO:30, SEQ ID NO:31 or SEQ ID NO:32.

3. The clk-2 gene of claim 1, which codes for a CLK-2 protein having the amino acid sequence set forth in SEQ ID NO:2.

4. The use of a clk-2 gene to alter function at the level of cellular physiology involved in the regulation of developmental rates, telomere length and/or longevity, wherein clk-2 mutations cause a longer life, altered cellular metabolism and physiological rates and an altered telomere length relative to the wild type, wherein clk-2 overexpression leads to telomere shortening, and wherein said gene codes for a protein having a sequence as set forth in SEQ ID NO:32.

5. A CLK-2 protein which has a function at the level of cellular physiology involved in the regulation of developmental rate, telomere length and longevity, wherein said CLK-2 protein is encoded by the gene of claim 1.

6. Use of a CLK-2 protein to alter a function at the level of cellular physiology involved in the regulation of developmental rate, telomere length and longevity, wherein clk-2 overexpression leads to telomere shortening, and wherein said CLK-2 protein is encoded by a gene as defined in claim 2.

7. A mutant CLK-2 protein which has the amino acid sequence set forth in SEQ ID NO:31.

8. A CLK-2 protein which has the amino acid sequence set forth in SEQ ID NO:2.

9. Use of CLK-2 protein to alter a function at the level of cellular physiology involved in the regulation of developmental rates, telomere length and longevity, wherein said CLK-2 protein has the amino acid sequence as set forth in SEQ ID NO:2, SEQ ID NO:3, SEQ ID NO:8, SEQ ID NO:9, SEQ ID NO:10, SEQ ID NO:11, SEQ ID NO:12, SEQ ID NO:13, SEQ ID NO:14, SEQ ID NO:17, SEQ ID NO:18, SEQ ID NO:19, SEQ ID NO:25, SEQ ID NO:26, SEQ ID NO:27, SEQ ID NO:28, SEQ ID NO:29, SEQ ID NO:30, SEQ ID NO:31 or SEQ ID NO:32.

10. A clk-2 gene which has the nucleotide sequence set forth in SEQ ID NO:1.

11. A mouse which comprises a gene knockout of the murine clk-2 gene homologue to a clk-2 gene as defined in claim 2.

12. A method to increase the life span of multicellular organism which comprises altering the function of telomeres and/or regulating telomere length.

13. The method of claim 12, wherein said multicellular organism is a metazoan.

14. A method to increase the life span of multicellular organism which comprises altering the mechanisms of sub-telomeric silencing and/or regulating telomere length.

15. The method of claim 14, wherein said multicellular organism is a metazoan.

16. The use of clk-2 gene as defined in claim 1, 3 or 10 and homologues thereof, to manipulate the physiological rates and/or telomere biology, whereby life span of an organism is altered.

17. Use of elk-2 gene as defined in claim 1, 3 or 10, or CLK-2 protein as defined in claim 5, 7 or 8 and homologues thereof, for screening drugs which decrease or increase the life span of a multicellular organism.

18. The use of claim 17, wherein said drug enhances or suppresses the expression of the clk-2 gene or activity of the protein CLK-2, and homologues thereof.

19. Use of a compound for the manufacture of a medicament for increasing and/or decreasing physiological rates of tissues, organ, and/or whole organism of a host; wherein said compound is interfering with activity of CLK-2 protein of claim 5, 7 or 8, and homologues thereof.

20. Use of a compound which promotes tissue and/or organ specific reduction or increase of clk-2 activity for the manufacture of a medicament for the treatment of pathological conditions causing increase of physiological rate of tissue and/or organ in an individual, wherein said compound is interfering with activity of CLK-2 protein of claim 5, 7 or 8, and homologues thereof.

21. Use of a compound which promotes tissue and/or organ specific reduction or increase of clk-2 activity for the manufacture of a medicament for the treatment of pathological conditions causing decrease of physiological rate of tissue and/or organ in an individual, wherein said compound is interfering with activity of CLK-2 protein as defined in claim 5, 7 or 8, and homologues thereof.

22. A clk-2 co-expressed gene which comprises a cex-7 gene having the nucleotide sequence set forth in SEQ ID NO:33 which codes for a CEX-7 protein having the amino acid sequence set forth in SEQ ID NO:34 wherein said gene is located in the clk-2 operon and said cex-7 gene is transcriptionally co-expressed with clk-2 gene present in said operon.

23. A human homologue of cex-7 gene of claim 22, wherein said gene codes for a protein having a sequence as set forth in SEQ ID NO:35.

24. Use of a human homologue of cex-7 gene of claim 22 and homologues thereof, to alter a function at the level of cellular level physiology involved in the regulation of developmental rates and longevity wherein said gene codes for a protein having a sequence as set forth in SEQ ID NO:35.

25. A mouse which comprises a gene knock out of the murine cex-7 gene homologue of the human gene as set forth in SEQ ID NO:35.

26. Use of a compound for the manufacture of a medicament for increasing and/or decreasing physiological rates of tissues, organ, and/or whole organism of a host; wherein said compound is interfering with activity of CEX-7 as defined in claim 22 or 23, and homologues thereof.

27. Use of a compound which promotes tissue and/or organ specific reduction or increase of cex-7 activity for the manufacture of a medicament for the treatment of pathological conditions causing increase of physiological rate of tissue and/or organ in an individual, wherein said compound is interfering with activity of CEX-7 as defined in claim 22 or 23, and homologues thereof.

28. Use of a compound which promotes tissue and/or organ specific reduction or increase of cex-7 activity for the manufacture of a medicament for the treatment of pathological conditions causing decrease of physiological rate of tissue and/or organ in an individual, wherein said compound is interfering with activity of CEX-7 as defined in claim 22 or 23, and homologues thereof.

29. A coq-4 gene which has a function at the level of cellular physiology involved in the regulation of. developmental rate and longevity, wherein coq-4 mutations cause altered cellular metabolism and physiological relative to the wild type, wherein coq-4 gene has the identifying characteristics of nucleotide sequence set forth in SEQ ID NO:36.

30. A coq-4 gene which has a function at the level of cellular physiology involved in the regulation of developmental rate and longevity, wherein coq-4 mutations cause altered cellular metabolism and physiological relative to the wild type, wherein coq-4 gene has the identifying characteristics of nucleotide sequence set forth in SEQ ID NO:36, wherein said gene codes for a protein having a sequence as set forth in SEQ ID NO:37.

31. Use of coq-4 gene to alter a function at the level of cellular physiology involved in the regulation of developmental rates, wherein coq-4 mutations cause an altered cellular metabolism and physiological rates relative to the wild type, wherein said gene codes for a protein having a sequence as set forth in SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51 or SEQ ID NO:52 and homologues thereof.

32. A COQ-4 protein which has a function at the level of cellular physiology involved in the regulation of developmental rate and longevity, wherein said COQ-4 protein is encoded by the gene of claim 29.

33. A mouse which comprises a gene knock out of the murine coq-4 gene as set forth in SEQ ID NO:45.

34. Use of a compound for the manufacture of a medicament for increasing and/or decreasing physiological rates of tissues, organs and/or whole organism of a host; wherein said compound is interfering with activity of COQ-4 protein as defined in claim 32, and homologues thereof.

35. Use of a compound which promotes tissue and/or organ specific reduction or increase of coq-4 activity for the manufacture of a medicament for the treatment of pathological conditions causing increase of physiological rate of tissue and/or organ in an individual, wherein said compound is interfering with activity of COQ-4 protein as defined in claim 32, and homologues thereof.

36. Use of a compound which promotes tissue and/or organ specific reduction or increase of coq-4 activity for the manufacture of a medicament for the treatment of pathological conditions causing decrease of physiological rate of tissue and/or organ in an individual, wherein said compound is interfering with activity of COQ-4 protein as defined in claim 32, and homologues thereof.

37. Use of a compound which promotes tissue and/or organ specific reduction or increase of clk-2 activity for the manufacture of a medicament for the treatment of pathological conditions due to altered telomere length in tissue and/or organ in an individual, wherein said compound is interfering with activity of CLK-2 protein as defined in claim 5, 7 or 8, and homologues thereof.
